(19) United States
(12) Patent Application Publication
(10) Pub. No.: US 2026/0148833 A1
(43) Pub. Date: May 28, 2026

(54) APPARATUS FOR AND METHOD OF GENERATING A REFRESHMENT HASH

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 19/431,263

(22) Filed: Dec. 23, 2025

Related U.S. Application Data
(63) Continuation-in-part of application No. 18/962,335, filed on Nov. 27, 2024, now Pat. No. 12,530,335.

(51) Int. Cl. G16H 20/60; A23L 33/00; G06F 16/22

(52) U.S. Cl. CPC G16H 20/60; A23L 33/30; G06F 16/2255

(57) ABSTRACT

An apparatus for generating a refreshment hash, wherein the apparatus include at least a processor, and a memory communicatively connected to the at least a processor, wherein the memory containing instructions configuring the at least a processor to receive a refreshment option; determine an individual biological profile associated with a user; identify a plurality of critical daily essential nutrients as a function of the determined individual biological profile; generate an updated refreshment option; calculate a nourishment label for the updated refreshment option using a dynamic scoring model; iteratively update the updated refreshment option; assign a nutrient badge to the updated refreshment option as a function of the nourishment label and one or more eligibility criteria determined as a function of the dynamic scoring model; and generate a refreshment hash as function of at least the updated refreshment option, the nourishment label, and the nutrient badge.

### CROSS-REFERENCE TO RELATED APPLICATIONS

This application is a continuation-in-part of U.S. Non-provisional patent application Ser. No. 18/962,335 filed on Nov. 27, 2024, and entitled “APPARATUS AND METHOD FOR GENERATING A REFRESHMENT HASH,” the entirety of which is incorporated herein by reference.

### FIELD OF THE INVENTION

The present invention generally relates to the field of nourishment dynamics. In particular, the present invention is directed to an apparatus for and method of generating a refreshment hash.

### BACKGROUND

Current refreshment options provide few options adjusted for nourishment indicators. Additionally, current refreshment options lack storage hash.

### SUMMARY OF THE DISCLOSURE

In an aspect, an apparatus for generating a refreshment hash, wherein the apparatus include at least a processor, and a memory communicatively connected to the at least a processor, wherein the memory containing instructions configuring the at least a processor to receive a refreshment option; determine an individual biological profile associated with a user, wherein the individual biological profile is determined as a function of a phenotypic cluster; identify a plurality of critical daily essential nutrients as a function of the determined individual biological profile; generate an updated refreshment option by adjusting at least an ingredient of the refreshment option as a function of the individual biological profile and the plurality of critical daily essential nutrients; calculate a nourishment label for the updated refreshment option using a dynamic scoring model, wherein the dynamic scoring model is configured to evaluate a nutrient intake; iteratively update the updated refreshment option by evaluating biological utilization data associated with consumption of the updated refreshment option; assign a nutrient badge to the updated refreshment option as a function of the nourishment label and one or more eligibility criteria determined as a function of the dynamic scoring model; and generate a refreshment hash as function of at least the updated refreshment option, the nourishment label, and the nutrient badge.

In yet another non-limiting aspect, a method of generating a refreshment hash, wherein the method include receiving, by at least a processor, a refreshment option; determining, by the at least a processor, an individual biological profile associated with a user, wherein the individual biological profile is determined as a function of a phenotypic cluster; identifying, by the at least a processor, a plurality of critical daily essential nutrients as a function of the determined individual biological profile; generating, by the at least a processor, an updated refreshment option by adjusting at least an ingredient of the refreshment option as a function of the individual biological profile and the plurality of critical daily essential nutrients; calculating, by the at least a processor, a nourishment label for the updated refreshment option using a dynamic scoring model, wherein the dynamic scoring model is configured to evaluate a nutrient intake; iteratively updating, by the at least a processor, the updated refreshment option by evaluating biological utilization data associated with consumption of the updated refreshment option; assigning, by the at least a processor, a nutrient badge to the updated refreshment option as a function of the nourishment label and one or more eligibility criteria determined as a function of the dynamic scoring model; and generating, by the at least a processor, a refreshment hash as function of at least the updated refreshment option, the nourishment label, and the nutrient badge.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A-B are an illustration of an exemplary embodiment of an apparatus for generating a refreshment hash;

FIG. 2 is an illustration of an exemplary embodiment of a machine learning module;

FIG. 3 is an illustration of an exemplary embodiment of a neural network;

FIG. 4 is an illustration of an exemplary embodiment of a node;

FIG. 5 is an illustration of an exemplary embodiment of an immutable sequential listing;

FIG. 6 is an illustration of an exemplary embodiment of a flow diagram of a method of generating a refreshment hash; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

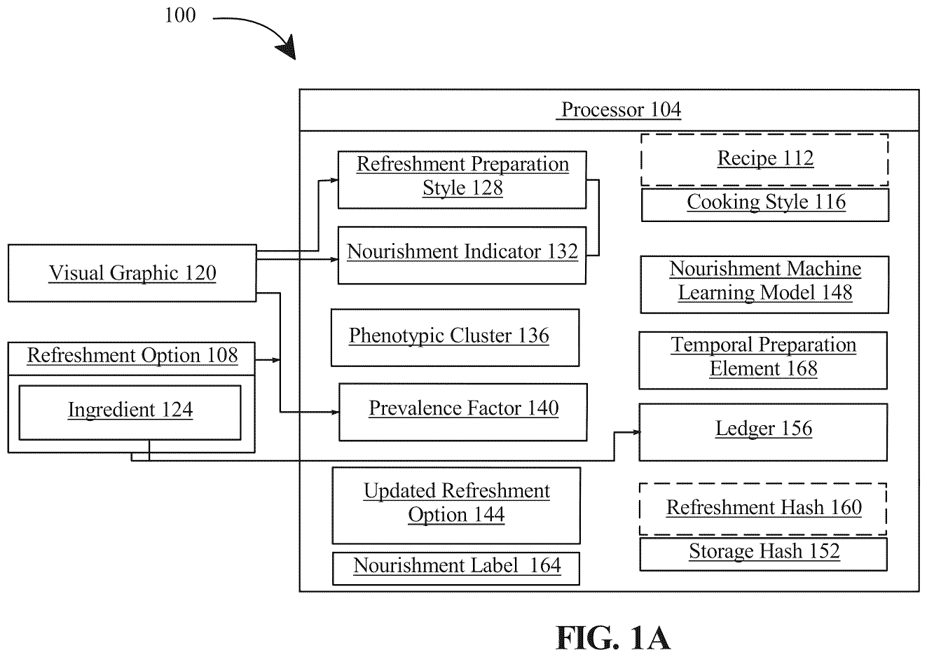

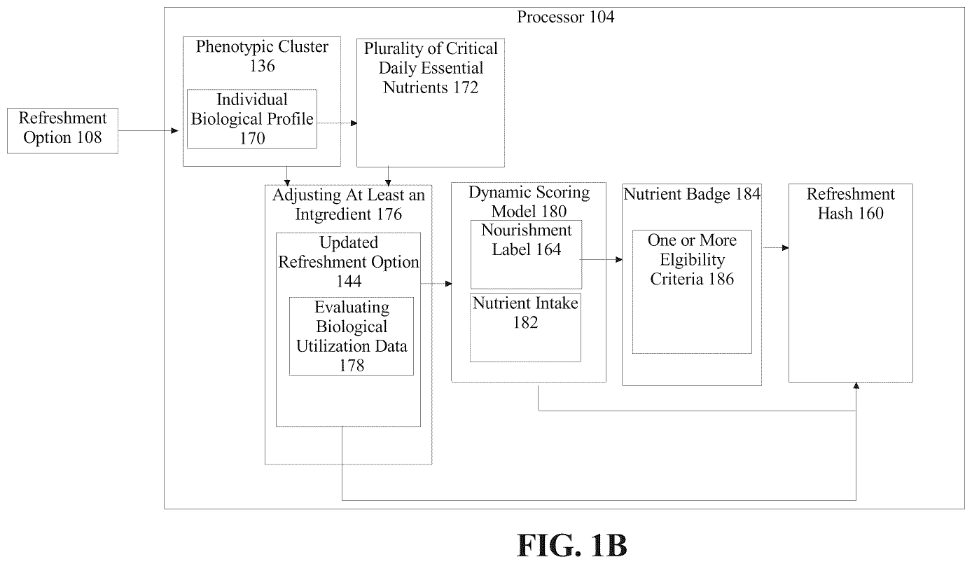

Referring now to FIGS. 1A-B, an exemplary embodiment of an apparatus 100 for generating a refreshment hash is illustrated. Processor 104 may include any computing device as described in this disclosure, including without limitation a microcontroller, microprocessor 104, digital signal processor 104 (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of system or apparatus 100 and/or computing device.

With continued reference to FIGS. 1A-B, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor 104 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIGS. 1A-B, as used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, apparatus, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example, and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, without limitation, via a bus or other facility for intercommunication between elements of a computing device. Communicative connecting may also include indirect connections via, for example, and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIGS. 1A-B, processor 104 is configured to receive a refreshment option 108. A “refreshment option” as used in this disclosure is any food and/or beverage available for consumption by a human being. A refreshment option 108 may include a recipe, a meal such as: breakfast; lunch; dinner; and/or a snack, a fruit, a vegetable, a grain, a cereal, meat, poultry, fish, seafood, dairy products, eggs, legumes, beans, nuts, seeds, oils, fats, baked goods, snack, confectionary, prepared food, plant based alternatives, fermented foods, herbs, spices, beverages, any combination thereof and the like. For instance and without limitation, a refreshment option 108 may include a combination of one or more of the following apples, bananas, berries, oranges, mangoes, grapes, spinach, carrots, potatoes, broccoli, tomatoes, lettuce, brown rice, quinoa, oats, barley, millet, whole wheat, white rice, white bread, pasta, beef, pork, lamb, chicken, turkey, duck, salmon, tuna, cod, tilapia, sardines, shrimp, crab, lobster, clams, oysters, milk, cheese, yogurt, butter, cream, chicken eggs, duck eggs, quail eggs, lentils, chickpeas, kidney beans, black beans, peas, soybeans, almonds, walnuts, cashews, peanuts, chia seeds, flax seeds, sunflower seeds, olive oil, coconut oil, butter, ghee, lard, margarine, bread, cakes, pastries, cookies, pies, chips, popcorn, chocolate, candy, ice cream, pizza, burgers, sandwiches, soups, salads, sushi, tofu, tempeh, seitan, plant-based burgers, almond milk, soy milk, coconut yogurt, yogurt, kimchi, sauerkraut, kombucha, pickles, basil, rosemary, turmeric, ginger, cinnamon, garlic, pepper, water, juices, tea, coffee, smoothies, soda, wine, beer, and the like. Refreshment option 108 may range in terms of nutrition, taste, preparation method, quantity, ingredient quality, and the like.

With continued reference to FIGS. 1A-B, refreshment option 108 includes a recipe 112. A “recipe” as used in this disclosure is any set of instructions and/or guidelines used to prepare and/or cook any meal, product and/or food substance intended for human consumption, dish, refreshment option 108 and the like. A recipe 112 may include a list of ingredients needed, quantities or measurements of each ingredient, and instructions on how to cook and/or prepare any meal, product and/or food substance intended for human consumption, dish, refreshment option 108 and the like. A recipe 112 may include information regarding cooking times, temperatures, and/or serving suggestions. A recipe 112 may include meal enhancements and instructions on alternative preparation methods. A recipe 112 may related to a particular cooking style 116 such as a recipe 112 for steak served with green beans and potatoes may relate to a paleo cooking style 116 whereas a recipe 112 for orecchiette pasta with broccoli sauce may relate to a vegetarian cooking style 116. A “cooking style” as used in this disclosure is any way or approach to selecting, preparing, and/or consuming food based on one or more dietary preferences. A cooking style 116 may emphasize certain food groups while avoiding others and may be guided by principles relating to nutrition, health conditions, personal values, and the like. For example, a cooking style 116 may be paleo diet, gluten free, vegetarian diet, vegan diet, ketogenic diet, mediterranean diet, low carb diet, Whole30 diet, DASH diet, raw food diet, and the like.

With continued reference to FIGS. 1A-B, refreshment option 108 includes a visual graphic 120. A “visual graphic” as used in this disclosure, is any visual representation of a refreshment option 108. A visual graphic 120 may include a drawing, painting, photograph, image, and the like. A visual graphic 120 may include a bar code such as a scannable pattern that encodes data. A bar code may include a machine-readable symptom, that stores information about a refreshment option 108 such as ingredients, nutritional content, cooking instructions, ingredient quantity, and the like. A bar code when scanned by a barcode reader, camera, smartphone app, and the like may be used to retrieve and/or process encoded information.

With continued reference to FIGS. 1A-B, processor 104 analyzes refreshment option 108 to identify at least an ingredient 124. An “ingredient” as used in this disclosure, is any substance and/or component used to make a refreshment option 108. An ingredient 124 may be found in its original raw state such as a macadamia nut or an avocado. An ingredient 124 may be processed such as white sugar which may undergo several stages of processing to exact sugar, purify it, and remove natural molasses resulting in white sugar. For example, a refreshment option 108 that indicates “fish tacos” may be analyzed by processor 104 to identify ingredient 124 that include halibut, corn tortillas containing corn flour, cabbage slaw containing green cabbage, purple cabbage, lime juice, and mayonnaise, guacamole containing avocados, cumin, lime juice, cilantro, black pepper, and paprika, fish seasoning containing cumin, paprika, garlic powder, chili powder, salt, pepper, and lime wedges. In an embodiment, an ingredient 124 may be identified from a source, indicating how the ingredient 124 was produced, raised, processed and the like. For example, an ingredient 124 may be indicated to be organic, free range, grass fed, grass finished, cage free, non-genetically modified organisms (GMO), conventional, farm-raised, wild-caught, locally grown, sustainable sourced, fair trade, hormone free, natural, pasture raised, and the like.

With continued reference to FIGS. 1A-B, ingredient 124 indicates a refreshment preparation style 128. A “refreshment preparation style” as used in this disclosure, is any preparation method utilized to create and/or prepare a refreshment option 108 and/or an ingredient 124. A refreshment preparation style 128 may impact flavor, texture, and overall quality of refreshment option 108. A refreshment preparation style 128 may indicate for example, chopping, dicing, slicing, mincing, grating, peeling, marinating, blanching, roasting, baking, frying, grilling, steaming, boiling, simmering, poaching, braising, stewing, sauteing, pressure cooking, sous vide, smoking, fermenting, freezing, and the like. A refreshment preparation style 128 may be labeled for an individual ingredient 124 and/or a combination of one or more ingredient 124 contained within a refreshment option 108. For instance and without limitation, a refreshment option 108 such as a meal containing millet porridge with stewed apples and hazelnuts may contain a refreshment preparation style 128 for the stewed apples, the hazelnuts, and the overall finished product of the millet porridge. In such an instance, the stewed apples may be prepared by peeling the apples, combining the apples in a large saucepan with sugar, lemon juice, and spices, cooking the apples over medium heat, and adjusting consistency to make the apples thicker if prepared. The hazelnuts may be prepared by roasting raw hazelnuts in an oven at 350 Reci for 10-15 minutes. The millet porridge may be prepared by combining millet with water or broth and gently simmering on a stovetop for 15-20 minutes. The final product may be prepared by topping a serving of millet porridge with the stewed apples and roasted hazelnuts.

With continued reference to FIGS. 1A-B, processor 104 receives a nourishment indicator 132. A “nourishment indicator” as used in this disclosure, is any demand for nourishment. A demand for nourishment may include any quantity of nourishment that may be requested at any given time. For example, a demand for nourishment may indicate a request at a restaurant to generate 35 entrees for dinner. In yet another non-limiting example, a demand for nourishment may indicate a request to produce on average 75 lunches. A demand for nourishment may be based on actual orders for nourishment and/or a number value that indicates an average and/or approximation based on a projected number of orders for a period of time such as during the course of a day, a meal, an hour, a week, a month, a season, and the like. A nourishment indicator 132 may be self-reported by a producer. A “producer” as used in this disclosure is any nourishment facility that prepares a refreshment option 108. A producer may include but is not limited to a grocery store, a restaurant, a chef, a kitchen, a café, a food truck, a cafeteria, a bakery, a deli, a catering service, a pop up restaurant, a meal kit producer, a dining facility, and the like. A producer may include any facility that produces food for consumption. A nourishment indicator 132 may be based on a certain number of meals, snacks, and/or ingredient 124 that a producer expects to produce and/or sell within a specified time frame. For example, a producer such as a grocery store may report a nourishment indicator 132 of 20 pre-packaged breakfasts that the grocery store aims to sell during a Tuesday lunch hour from 11 am-2 μm. In yet another non-limiting example, a producer such as a meal preparation company may report a nourishment indicator 132 of 1000 dinners that they prepare on average in a given week. A nourishment indicator 132 may specify information relating to phenotypic clusters and demand for nourishment based on clusters. For instance and without limitation, a nourishment indicator 132 may specify that 25% of the demand is for a lunch option for individuals who have autoimmune conditions. In yet another non-limiting example, a nourishment indicator 132 may specify that 50 dinners need to be prepared for users who have insulin resistance. In yet another non-limiting example, a nourishment indicator 132 may specify that 30% of the population is African American and at risk of developing hypertension. A nourishment indicator 132 May specify information relating to a particular eating style such that 50% of the demand is for meals that are gluten free or 75% of the demand is for meals that are vegetarian.

With continued reference to FIGS. 1A-B, nourishment indicator 132 identifies a phenotypic cluster 136 located within a geographic location. A “phenotype,” as used in this disclosure, is a composite observable characteristic or trait of an individual. A phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Phenotype may include a congenital disorder, anomaly, and the like, such as hearing defects, trisomy 18 (Edward's syndrome), trisomy 21 (down syndrome), trisomy 13 (Patau syndrome), cleft palate, spina bifida, phenylketonuria, glutamate carboxypeptidase II mutation, pyloric stenosis, congenital hip dislocation, anencephaly, hypoplasia, Meckel's diverticulum, and the like. Phenotype may include a genotype-environment interaction (GxE). Phenotype may include any diagnosis (current disorder) and/or prognosis (predicted difficulty, future diagnosis, outcome, and the like) associated with a congenital factor. Phenotype may include identifiers associated with disorders, conditions, symptoms, and the like, which may correspond with categorization. Phenotype may include a predictive classification, where a subject may be considered reasonably healthy at birth, does not harbor congenital factor(s) indicative of obvious current congenital disorder but may include data that indicates a phenotype with which they may be most closely categorized to, and/or an imminent categorization. A phenotype may be stored and/or retrieved from a user database. A “phenotypic cluster” as used in this disclosure, is a group of one or more phenotypes located within a specified geographical location. A “geographical location” as used in this disclosure is any boundary defined by a map, GPS, RFID, wi-fi, cellular data and the like. For example, a geographical location may include a specific location such as a shopping center, or a restaurant. A geographical location may include a location such as Denver, Colorado, a neighborhood such as Back Bay in Boston, Massachusetts, a zip code such as 02114, a town such as Savannah, Georgia, and the like. A phenotypic cluster 136 contains a prevalence factor 140. A “prevalence factor” as used in this disclosure, is any proportion of a population within a geographical location that have a specific characteristic, condition, and/or phenotype at a given time. A prevalence factor 140 may be expressed as a percentage or fraction such as the prevalence of influenza within a geographical location may be 20%. A phenotypic cluster 136 within a geographical location may specify for example a prevalence of individuals with Type Two Diabetes Mellitus located within Atlanta, Georgia. A phenotype and/or phenotype cluster may include any phenotype and/or phenotype cluster as described in U.S. patent application Ser. No. 18/090,411 filed on Dec. 28, 2022 and entitled “APPARATUS AND METHOD FOR SCORING A NUTRIENT” and U.S. patent application Ser. No. 17/976,329 filed on Oct. 28, 2022 and entitled “APPARATUS AND METHOD FOR GENERATING AN INGREDIENT 124 CHAIN” both of which are incorporated by reference in their entirety.

With continued reference to FIGS. 1A-B, in some embodiments, nourishment indicator 132 may further identify an individual biological profile associated with a specific user, wherein the individual biological profile 170 refines or supplements a phenotypic cluster 136 to account for individualized biological needs. An “individual biological profile,” as used in this disclosure, is a data construct representing one or more individualized biological states, requirements, or constraints of a user that influence nourishment suitability. An individual biological profile 170 may include, without limitation, individualized biochemical markers, metabolic characteristics, micronutrient deficiencies or excesses, hydration status, inflammatory markers, hormonal profiles, allergy or intolerance indicators, digestive or absorption efficiency, medication interactions, activity level, age, sex, body composition, pregnancy or lactation status, recovery state, or combinations thereof. In some embodiments, the individual biological profile 170 may include data derived from laboratory testing, wearable devices, user-reported inputs, electronic health records, historical nourishment consumption data, or inferred biological states generated by one or more machine-learning models. The individual biological profile 170 may be static or dynamic and may be updated over time as a function of newly received biological data, environmental factors, or behavioral inputs. In such embodiments, processor 104 may utilize the individual biological profile 170 in conjunction with, or in lieu of, the phenotypic cluster 136 and prevalence factor 140 to adjust at least an ingredient 124, generate an updated refreshment option 144, or calculate a nourishment label 164 that is tailored to the individualized biological needs of the user while remaining consistent with broader population-level phenotypic constraints.

With continued reference to FIGS. 1A-B, a geographical location may include a geofence. As used in the current disclosures, a “geofence” is a virtual perimeter or boundary defined by geographic coordinates in a digital mapping system. Geographical coordinates may include a radius from a geographical point, proximity to a landmark, zip codes, area codes, longitude and latitude, cities, states, countries, counties, travel time, and the like. A geofence may be generated as a radius around a point or location or arbitrary borders drawn by a user. In some embodiments, the point or location may be selected by a user through user input, wherein user input may include, as non-limiting examples, tapping on a screen, inputting an address, inputting coordinates, and the like. A geofence may be generated to match a predetermined set of boundaries such as neighborhoods, school zones, zip codes, county, state, and city limits, area codes, voting districts, geographic regions, streets, rivers, other landmarks, and the like. In embodiments, geofences may be generated as a function of a user input. Geofences may be used in location-based services and applications to trigger specific actions or events when a mobile device or GPS-enabled object enters, exits, or remains within the designated area. In an embodiment, a geofence may include a time-based geofence. In addition to geographical boundaries, time-based geofences trigger events or notifications based on specific time intervals or schedules. For example, a geographic location may include a requirement that the user be physically located within the geofenced area for a specific duration such as one hour. A geofence may include a proximity geofence. As used in the current disclosure, a “proximity geofence” is a virtual boundary or area defined by geographical coordinates which is used to trigger specific actions or events. This may include events such as when a mobile device or object enters or exits that is outlined by the proximity based geofence. Proximity geofences are used to trigger events when a device or location is within or near the vicinity of a specific location. They are often used for location-based marketing and notifications. This may be used to let a user know when they are nearing the boundary for the geofenced area. For an example, processor 104 may send a user a notification stating that if they were to move into a nearby geofenced area the user may be eligible to be hired by the entity. In additional embodiment, a geofence may include a dynamic geofence. Dynamic geofences can change in real-time based on variables like a user's location, device data, environmental factors, employer preferences, and the like. This allows for adaptive and context-aware geofencing applications.

With continued reference to FIGS. 1A-B, in some embodiments, nourishment indicator 132 and/or an individual biological profile 170 may further incorporate a set of critical daily essential nutrients defining baseline nourishment requirements necessary to support physiological function over a defined temporal interval. As used in this disclosure, “critical daily essential nutrients” are nutrients, compounds, or hydration requirements that an individual should obtain on a recurring basis to maintain biological viability, metabolic stability, or functional health. Critical daily essential nutrients 172 may include, without limitation, macronutrients, micronutrients, electrolytes, amino acids, fatty acids, vitamins, minerals, water intake requirements, or combinations thereof, and may further include minimum intake thresholds, maximum intake thresholds, target ranges, or proportional distributions specific to an individual biological profile 170, phenotypic cluster 136, or population subgroup.

In some embodiments, processor 104 may determine critical daily essential nutrients 172 as a function of individualized biological data, phenotypic characteristics, environmental conditions, activity level, age, sex, metabolic demand, or inferred biological state. The set of critical daily essential nutrients 172 may be static or dynamically adjustable and may be updated over time based on changes to the individual biological profile 170, bioextraction assessment results, or observed nutrient utilization efficiency. Processor 104 may utilize the critical daily essential nutrients 172 as a baseline constraint when generating, scoring, or adjusting a refreshment option 108 or updated refreshment option 144, such that one or more recipes are configured to satisfy, partially satisfy, or collectively contribute toward fulfillment of the critical daily essential nutrient requirements over a defined time period. In such embodiments, critical daily essential nutrients 172 may be evaluated on a per-recipe basis, an aggregate daily basis, or across multiple refreshment options consumed within a temporal window, thereby enabling processor 104 to generate nourishment outputs that are not solely optimized for a single refreshment option, but are coordinated to achieve cumulative daily biological adequacy consistent with individualized nourishment objectives. As used in this disclosure, a biological state refers to a representational condition or status of one or more physiological, biochemical, or metabolic characteristics of an individual at a given time or over a temporal interval. A biological state may reflect current, inferred, or predicted biological conditions relevant to nutrient utilization, hydration, metabolic demand, or overall biological alignment with nourishment inputs, without requiring medical diagnosis or clinical assessment. In some embodiments, a biological state may include indicators associated with nutrient sufficiency or deficiency, metabolic efficiency, inflammatory response, hydration balance, recovery or exertion level, or other biologically relevant parameters derived from observed or inferred data. In embodiments utilizing a machine learning model, a predicted biological state may be generated by analyzing historical biological utilization data, nutrient intake patterns, individual biological profile 170s, phenotypic cluster data, or combinations thereof, to estimate a likely biological condition of an individual at a current or future time. The predicted biological state may be represented as a classification, score, vector, probability distribution, or other data structure and may be used as an input to a dynamic scoring model, eligibility criteria evaluation, or refreshment option adjustment process.

With continued reference to FIGS. 1A-B, processor 104 adjusts an ingredient 124 as a function of the nourishment indicator 132. “Adjusting an ingredient” as used in this disclosure, includes any change made to a refreshment option 108. A change to a refreshment option 108 may include changing the quantity of an ingredient 124, changing the method of preparing an ingredient 124 and/or refreshment option 108, changing the source of the ingredient 124, changing the sequence of when an ingredient 124 is incorporated into a refreshment option 108, substituting an ingredient 124, adding an ingredient 124, removing an ingredient 124 and the like. For example, processor 104 may adjust 176 an ingredient 124 such as sunflower oil for avocado oil. In yet another non-limiting example, processor 104 may adjust an ingredient 124 such as red kidney beans for crushed tomatoes. Processor 104 may adjust an ingredient 124 using nourishment indicator 132 and/or information relating to a phenotype and/or phenotype cluster. For instance and without limitation, a nourishment indicator 132 that specifies that a large percentage of demand has iron deficiency anemia may cause processor 104 to adjust iron rich foods to be paired with foods high in Vitamin C to maximize iron absorption. In yet another non-limiting example, a nourishment indicator 132 that specifies a demand for meals suitable for individuals with Hashimoto's thyroid disease may cause processor 104 to adjust meals to eliminate cruciferous vegetables such as cabbage, broccoli, cauliflower, kale, Brussel sprouts, and Bok choy, due to the ability of cruciferous vegetables to block absorption of iodine. Recommendations as to what ingredient 124 to adjust may be made by best practices platform, including any best practices platform and/or expert database as described in U.S. patent application Ser. No. 17/463,882, filed on Sep. 1, 2021 and entitled “METHODS AND SYSTEMS FOR CLASSIFICATION USING EXPERT DATA,” the entirety of which is incorporated herein by reference in its entirety.

With continued reference to FIGS. 1A-B, processor 104 outputs an updated refreshment option 144. An “updated refreshment option” as used in this disclosure, is any refreshment option 108 that has been adjusted. Adjusting, including any methodology as described above in reference to adjusting an ingredient 124. For instance and without limitation, a refreshment option 108 such as chicken fried in soybean oil may be adjusted whereby the chicken is fried in a more temperature stable oil such as coconut oil. In yet another non-limiting example, a refreshment option 108 such as coconut shrimp tacos may be adjusted where by the updated refreshment option 144 contains sautéed shrimp tacos. An updated refreshment option 144 may include an additional ingredient 124, an ingredient 124 that is substituted, removal of an ingredient 124, a different preparation style, an extra preparation step, removal of a preparation step, and the like. For example, a refreshment option 108 such as chicken noodle soup may be adjusted whereby the wheat durum noodles in the soup are substituted for gluten free corn noodles. In yet another non-limiting example, a refreshment option 108 such as friend haddock may be adjusted whereby the haddock is sauteed with olive oil in lieu of frying.

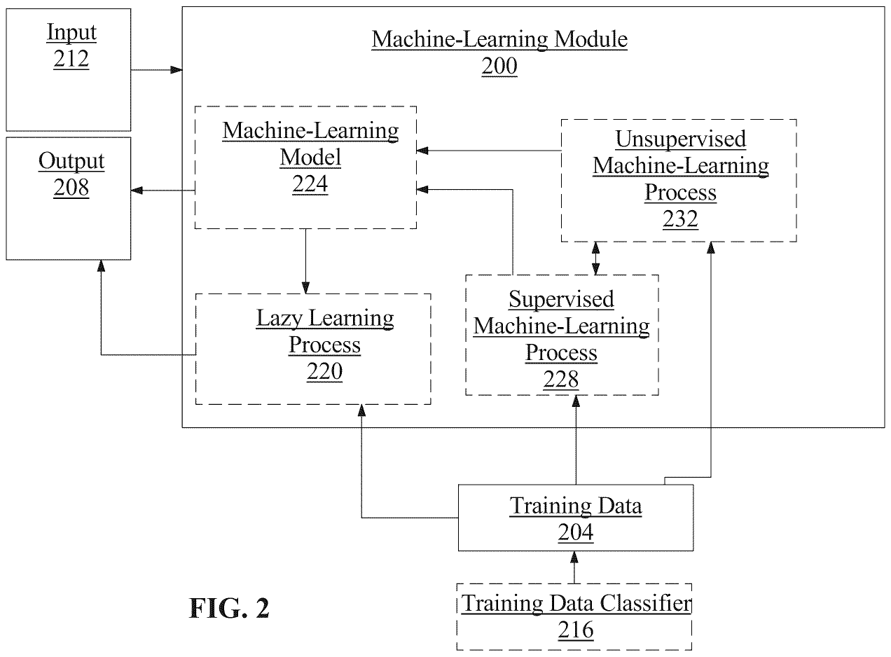

With continued reference to FIGS. 1A-B, adjusting an ingredient 124 may include generating a nourishment machine learning model 148. A machine learning model includes any machine learning model as described below in more detail in reference to FIGS. 2-4. Nourishment machine learning model 148 may utilize a refreshment option 108 as an input and output an updated refreshment option 144. For example, an input such as a recipe 112 for blueberry muffins made with whole wheat flour may be input to nourishment machine learning model 148 whereby output may be a recipe 112 for blueberry muffins made with collagen powder and spelt flour to increase protein content. In yet another non-limiting example, an input to nourishment machine learning model 148 such as a recipe 112 for chicken stir-fry with peas may be output as tofu stir-fry with peas. Refreshment option 108 may include a refreshment source. A “refreshment source” as used in this disclosure is any indicator as to the source of a refreshment option 108 and/or ingredient 124. A source may indicate the origin of a refreshment option 108 and/or ingredient 124. For example, a source may indicate that a carrot was grown using organic ingredient 124 from Wilson Farms located in Lexington, MA. In yet another non-limiting example, a source may indicate that chicken was obtain from a farm that raised the chicken using free-range conditions and being fed an organic grain free diet. A source may indicate the nutritional content of a refreshment source and/or ingredient 124. For example, a source may indicate that grass fed and finished flank steak has 1300 mg servings of omega-3 fatty acids per serving whereas a conventional flank steam may have 0 mg servings of omega-3 fatty acids and 600 mg of omega-6 fatty acids. A source may indicate where and/or how a refreshment option 108 may be assembled into a final product. For example, a refreshment option 108 consisting of a shredded chicken taco bowl may contain free range chicken grown in New Hampshire, spices sourced from an organic grocery store, avocados grown on an organic farm in California, Pico de Gallo made at a grocery store in California, and rice grown in Thailand and cooked in California. One or more ingredient 124 contained within a refreshment option 108 may indicate one or more sources of ingredient 124 and/or growing conditions of an ingredient 124. For instance in the above example, the Pico de Gallo may contain tomatoes sourced from Mexico, basil sourced from Italy, Cilantro sourced from Mexico, lime juice sourced from California, and onions sourced from South Dakota. Refreshment source of ingredient 124 and/or refreshment option 108 may include a source indicator, including any source as described herein.

With continued reference to FIGS. 1A-B, in some embodiments, the updated refreshment option 144 generated by nourishment machine learning model 148 may be configured as a continuously adaptive output that is subject to an ongoing biological evaluation loop, rather than a fixed or terminal result. In such embodiments, processor 104 may initiate a post-generation evaluation phase in which biological response data associated with consumption of the updated refreshment option 144 is monitored, received, inferred, or otherwise analyzed to determine an effectiveness of nutrient delivery relative to an individual biological profile 170. This evaluation phase may include performing a bioextraction assessment, wherein processor 104 evaluates an extent to which nutrients contained within one or more ingredients 124 are absorbed, metabolized, retained, or excreted by an individual over a period of time. Bioextraction assessment may further include analysis of nutrient bioavailability, digestive efficiency, interaction effects between ingredients, hydration response, metabolic rate variability, inflammatory response, or other physiological indicators indicative of nourishment utilization.

In some embodiments, the evaluation loop may further comprise comparing observed or inferred biological response data against one or more target biological thresholds, baseline requirements, or predicted outcomes associated with the individual biological profile 170, phenotypic cluster 136, or nourishment indicator 132. Where a deviation between expected and observed biological response is identified, processor 104 may generate a corrective adjustment signal configured to modify subsequent nourishment generation. Based on the corrective adjustment signal, processor 104 may re-generate nourishment machine learning model 148 to iteratively adjust one or more ingredients 124, ingredient quantities, refreshment preparation styles 128, refreshment sources, or combinations thereof, thereby generating a successive updated refreshment option 144. In such embodiments, the evaluation loop may be executed continuously, periodically, or event-triggered as a function of newly received biological data, behavioral inputs, environmental conditions, or temporal factors, enabling real-time or near real-time nourishment optimization. The iterative evaluation and adjustment loop may persist until one or more convergence criteria are satisfied, including attainment of desired biological response metrics, stabilization of nutrient utilization efficiency, or compliance with nourishment indicator 132 constraints, while maintaining compatibility with population-level phenotypic considerations.

With continued reference to FIGS. 1A-B, in some embodiments, processor 104 may be further configured to generate a nutrient badge 184 or scoring badge associated with an updated refreshment option 144, wherein the nutrient badge 184 represents a certification of alignment between the updated refreshment option 144 and one or more individualized or population-level biological nourishment criteria. As used in this disclosure, a “nutrient badge” is a data object, indicator, or designation assigned to a refreshment option 108 or updated refreshment option 144 based on satisfaction of defined nourishment scoring requirements derived from nourishment label 164, critical daily essential nutrient thresholds, individual biological profiles, phenotypic clusters 136, or combinations thereof. In some embodiments, processor 104 may determine eligibility of an updated refreshment option 144 to receive or maintain a nutrient badge 184 by evaluating the updated refreshment option 144 against a dynamic scoring framework, wherein one or more nutrient composition metrics, hydration-related factors, or ingredient characteristics are compared to individualized or group-based biological requirements. Eligibility determination may include verifying that the updated refreshment option 144 satisfies minimum scoring thresholds, nutrient balance constraints, or proportional intake targets while accounting for cumulative daily intake and ongoing biological evaluation. In such embodiments, processor 104 may generate different nutrient badges corresponding to different biological objectives, phenotypic groupings, or individual nourishment needs, wherein a single refreshment option 108 may qualify for one or more nutrient badges under different scoring contexts.

In further embodiments, nutrient badge 184 eligibility may be continuously monitored and dynamically updated as a function of changes to nourishment label 164, real-time nutrient or hydration intake data, bioextraction assessment results, or updates to an individual biological profile 170. Where updated refreshment option 144 no longer satisfies one or more eligibility criteria 186, processor 104 may revoke, modify, or withhold the nutrient badge 184, and may re-adjust one or more ingredients 124, preparation styles 128, or quantities to restore eligibility in a subsequent updated refreshment option 144. In such embodiments, the nutrient badge 184 operates as a dynamic certification mechanism that reflects ongoing compliance with biological nourishment requirements rather than a static designation, thereby protecting the process by which refreshment options are generated, evaluated, and maintained in alignment with individualized and population-level biological needs.

With continued reference to FIGS. 1A-B, in some embodiments, processor 104 may be further configured to generate a nutrient score associated with a refreshment option 108 or updated refreshment option 144, wherein the nutrient score represents a quantified evaluation of nutrient composition relative to biological requirements. As used in this disclosure, a “nutrient score” is a calculated value derived from analyzing one or more nutrients present in a refreshment option 108 in view of individualized biological needs, phenotypic characteristics, or critical daily essential nutrient requirements. The nutrient score may be generated by assigning weighted values to nutrients, nutrient groups, or hydration-related factors based on their relative contribution to biological adequacy, deficiency mitigation, or excess avoidance.

In some embodiments, processor 104 may generate the nutrient score by aggregating nutrient contribution values across one or more ingredients 124 contained within a refreshment option 108, wherein each nutrient contribution value is adjusted based on predicted bioavailability, preparation style 128, refreshment source, or inferred biological utilization 178 efficiency. The nutrient score may further incorporate penalties or reductions associated with nutrients exceeding maximum intake thresholds, competing nutrient interactions, or preparation-induced nutrient degradation. In such embodiments, the nutrient score represents a biologically contextualized assessment rather than a static nutritional total. As used in this disclosure, biological utilization data refers to data indicative of how nutrients contained within a refreshment option 108 or updated refreshment option 144 are biologically processed, absorbed, metabolized, retained, or otherwise utilized by an individual over time. Biological utilization data 178 may include measured, inferred, estimated, or predicted indicators of nutrient absorption efficiency, bioavailability, metabolic utilization, hydration response, digestive processing, nutrient retention, or excretion patterns. In some embodiments, biological utilization data 178 may be obtained directly from sensors, wearable devices, laboratory measurements, or user-reported inputs, while in other embodiments biological utilization data may be inferred or predicted by one or more computational models or machine learning models based on historical consumption patterns, phenotypic characteristics, individual biological profiles, or aggregated population data. Biological utilization data 178 may be associated with a temporal interval following consumption of a refreshment option and may reflect short-term or longer-term utilization trends. In such embodiments, processor 104 may utilize biological utilization data as feedback for iteratively updating refreshment options, recalibrating a dynamic scoring model, adjusting nutrient scores or nourishment labels, or determining eligibility for a nutrient badge 184, without requiring direct medical diagnosis or treatment determination.

In further embodiments, the nutrient score may be calculated dynamically as a function of cumulative nutrient intake across multiple refreshment options consumed within a defined temporal window, and may be updated in real time or near real time based on newly received intake data, hydration status, or biological response indicators. Processor 104 may utilize the nutrient score as an intermediate or component value for calculating the nourishment label 164, determining eligibility for a nutrient badge 184, or triggering adjustment of one or more ingredients 124 in a subsequent updated refreshment option 144.

With continued reference to FIGS. 1A-B, in some embodiments, generating the nutrient score may comprise executing a multi-stage nutrient scoring process configured to quantify biological contribution of nutrients contained within a refreshment option 108 or updated refreshment option 144. In such embodiments, processor 104 may identify a plurality of nutrients present in one or more ingredients 124, wherein each nutrient is associated with a corresponding quantity derived from ingredient composition data, portion size, or preparation parameters. For each identified nutrient, processor 104 may determine a nutrient contribution value representing an estimated biological contribution of the nutrient, wherein the nutrient contribution value is calculated as a function of nutrient quantity, predicted bioavailability, preparation style 128, refreshment source characteristics, and inferred nutrient utilization efficiency associated with an individual biological profile 170.

In some embodiments, processor 104 may apply one or more weighting factors to the nutrient contribution values, wherein weighting factors are derived from critical daily essential nutrient requirements, individualized biological needs, phenotypic cluster constraints, or combinations thereof. Weighting factors may increase, decrease, or normalize nutrient contribution values based on whether a nutrient is underrepresented, adequately represented, or overrepresented relative to target intake ranges. Processor 104 may further apply constraint logic to cap, scale, or penalize nutrient contribution values associated with nutrients exceeding maximum intake thresholds or producing predicted adverse biological interactions. In such embodiments, processor 104 may aggregate the weighted nutrient contribution values to generate a composite nutrient score, wherein aggregation may include summation, normalization, proportional scaling, or other mathematical combination techniques. The nutrient score may be calculated for a single refreshment option, or may be calculated cumulatively across multiple refreshment options consumed within a defined temporal window. In further embodiments, weighting factors and constraint parameters used to generate the nutrient score may be dynamically adjusted over time based on observed biological response data, bio extraction assessment results, or deviations from critical daily essential nutrient requirements, thereby enabling adaptive and biologically contextualized nutrient scoring. As used in this disclosure, a contribution value refers to a quantified representation of a relative biological impact of an individual nutrient contained within an updated refreshment option 144. In some embodiments, a contribution value may be determined for each identified nutrient as a function of one or more factors including nutrient quantity, predicted bioavailability, preparation style, refreshment source characteristics, and inferred biological utilization efficiency associated with an individual biological profile 170. A contribution value may represent a positive contribution, a neutral contribution, or a reduced contribution based on whether the nutrient supports, has negligible effect on, or detracts from satisfaction of critical daily essential nutrient requirements.

In such embodiments, contribution values may be expressed as numerical values, normalized scores, weighted coefficients, vectors, or other data structures suitable for aggregation and comparison. Contribution values may further incorporate adjustments or penalties where nutrients exceed maximum intake thresholds, interact adversely with other nutrients, or experience degradation due to preparation or storage. The contribution values may serve as intermediate computational outputs that enable aggregation of nutrient effects across multiple nutrients to generate a composite nutrient score reflective of overall biological alignment. With continued reference to FIGS. 1A-B, processor 104 secures updated refreshment option 144 within a refreshment hash 160 as a function of a storage hash 152. A “hash,” as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

In an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger 156 as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-512,” and the like), Message Authentication Code (“MAC”)-family hash functions such as PMAC, OMAC, VMAC, HMAC, and UMAC, Poly1305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatún hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2^{n/2}) for n output bits; thus, it may take on the order of 2²⁵⁶ operations to locate a collision in a 512 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

Continuing to refer to FIGS. 1A-B, a “secure proof,” as used in this disclosure, is a protocol whereby an output is generated that demonstrates possession of a secret, such as device-specific secret, without demonstrating the entirety of the device-specific secret; in other words, a secure proof by itself, is insufficient to reconstruct the entire device-specific secret, enabling the production of at least another secure proof using at least a device-specific secret. A secure proof may be referred to as a “proof of possession” or “proof of knowledge” of a secret. Where at least a device-specific secret is a plurality of secrets, such as a plurality of challenge-response pairs, a secure proof may include an output that reveals the entirety of one of the plurality of secrets, but not all of the plurality of secrets; for instance, secure proof may be a response contained in one challenge-response pair. In an embodiment, proof may not be secure; in other words, proof may include a one-time revelation of at least a device-specific secret, for instance as used in a single challenge-response exchange.

Secure proof may include a zero-knowledge proof, which may provide an output demonstrating possession of a secret while revealing none of the secret to a recipient of the output; zero-knowledge proof may be information-theoretically secure, meaning that an entity with infinite computing power would be unable to determine secret from output. Alternatively, zero-knowledge proof may be computationally secure, meaning that determination of secret from output is computationally infeasible, for instance to the same extent that determination of a private key from a public key in a public key cryptographic system is computationally infeasible. Zero-knowledge proof algorithms may generally include a set of two algorithms, a prover algorithm, or “P,” which is used to prove computational integrity and/or possession of a secret, and a verifier algorithm, or “V” whereby a party may check the validity of P. Zero-knowledge proof may include an interactive zero-knowledge proof, wherein a party verifying the proof must directly interact with the proving party; for instance, the verifying and proving parties may be required to be online, or connected to the same network as each other, at the same time. Interactive zero-knowledge proof may include a “proof of knowledge” proof, such as a Schnorr algorithm for proof on knowledge of a discrete logarithm. In a Schnorr algorithm, a prover commits to a randomness r, generates a message based on r, and generates a message adding r to a challenge c multiplied by a discrete logarithm that the prover is able to calculate; verification is performed by the verifier who produced c by exponentiation, thus checking the validity of the discrete logarithm. Interactive zero-knowledge proofs may alternatively or additionally include sigma protocols. Persons skilled in the art, upon reviewing the entirety of this disclosure, will be aware of various alternative interactive zero-knowledge proofs that may be implemented consistently with this disclosure.

Alternatively, zero-knowledge proof may include a non-interactive zero-knowledge, proof, or a proof wherein neither party to the proof interacts with the other party to the proof; for instance, each of a party receiving the proof and a party providing the proof may receive a reference datum which the party providing the proof may modify or otherwise use to perform the proof. As a non-limiting example, zero-knowledge proof may include a succinct non-interactive arguments of knowledge (ZK-SNARKS) proof, wherein a “trusted setup” process creates proof and verification keys using secret (and subsequently discarded) information encoded using a public key cryptographic system, a prover runs a proving algorithm using the proving key and secret information available to the prover, and a verifier checks the proof using the verification key; public key cryptographic system may include RSA, elliptic curve cryptography, ElGamal, or any other suitable public key cryptographic system. Generation of trusted setup may be performed using a secure multiparty computation so that no one party has control of the totality of the secret information used in the trusted setup; as a result, if any one party generating the trusted setup is trustworthy, the secret information may be unrecoverable by malicious parties. As another non-limiting example, non-interactive zero-knowledge proof may include a Succinct Transparent Arguments of Knowledge (ZK-STARKS) zero-knowledge proof. In an embodiment, a ZK-STARKS proof includes a Merkle root of a Merkle tree representing evaluation of a secret computation at some number of points, which may be 1 billion points, plus Merkle branches representing evaluations at a set of randomly selected points of the number of points; verification may include determining that Merkle branches provided match the Merkle root, and that point verifications at those branches represent valid values, where validity is shown by demonstrating that all values belong to the same polynomial created by transforming the secret computation. In an embodiment, ZK-STARKS does not require a trusted setup.

Zero-knowledge proof may include any other suitable zero-knowledge proof. Zero-knowledge proof may include, without limitation bulletproofs. Zero-knowledge proof may include a homomorphic public-key cryptography (hPKC)-based proof. Zero-knowledge proof may include a discrete logarithmic problem (DLP) proof. Zero-knowledge proof may include a secure multi-party computation (MPC) proof. Zero-knowledge proof may include, without limitation, an incrementally verifiable computation (IVC). Zero-knowledge proof may include an interactive oracle proof (IOP). Zero-knowledge proof may include a proof based on the probabilistically checkable proof (PCP) theorem, including a linear PCP (LPCP) proof. Persons skilled in the art, upon reviewing the entirety of this disclosure, will be aware of various forms of zero-knowledge proofs that may be used, singly or in combination, consistently with this disclosure.

In an embodiment, secure proof is implemented using a challenge-response protocol. In an embodiment, this may function as a one-time pad implementation; for instance, a manufacturer or other trusted party may record a series of outputs (“responses”) produced by a device possessing secret information, given a series of corresponding inputs (“challenges”), and store them securely. In an embodiment, a challenge-response protocol may be combined with key generation. A single key may be used in one or more digital signatures as described in further detail below, such as signatures used to receive and/or transfer possession of crypto-currency assets; the key may be discarded for future use after a set period of time. In an embodiment, varied inputs include variations in local physical parameters, such as fluctuations in local electromagnetic fields, radiation, temperature, and the like, such that an almost limitless variety of private keys may be so generated. Secure proof may include encryption of a challenge to produce the response, indicating possession of a secret key. Encryption may be performed using a private key of a public key cryptographic system, or using a private key of a symmetric cryptographic system; for instance, trusted party may verify response by decrypting an encryption of challenge or of another datum using either a symmetric or public-key cryptographic system, verifying that a stored key matches the key used for encryption as a function of at least a device-specific secret. Keys may be generated by random variation in selection of prime numbers, for instance for the purposes of a cryptographic system such as RSA that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

Keys may be generated by random variation in selection of prime numbers, for instance for the purposes of a cryptographic system such as secret that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

Cryptographic system may be configured to generate a session-specific secret. Session-specific secret may include a secret, which may be generated according to any process as described above, that uniquely identifies a particular instance of an attested boot and/or loading of software monitor. Session-specific secret may include without limitation a random number. Session-specific secret may be converted to and/or added to a secure proof, verification datum, and/or key according to any process as described above for generation of a secure proof, verification datum, and/or key from a secret or “seed”; session-specific secret, a key produced therewith, verification datum produced therewith, and/or a secure proof produced therewith may be combined with module-specific secret, a key produced therewith, a verification datum produced therewith, and/or a secure proof produced therewith, such that, for instance, a software monitor and/or other signed element of attested boot and/or attested computing may include secure proof both of session-specific secret and of module-specific secret. In an embodiment, session-specific secret may be usable to identify that a given computation has been performed during a particular attested session, just as device-specific secret may be used to demonstrate that a particular computation has been produced by a particular device. This may be used, e.g., where secure computing module and/or any component thereof is stateless, such as where any such element has no memory that may be overwritten and/or corrupted.

A “digital signature,” as used herein, includes a secure proof of possession of a secret by a signing device, as performed on provided element of data, known as a “message.” A message may include an encrypted mathematical representation of a file or other set of data using the private key of a public key cryptographic system. Secure proof may include any form of secure proof as described above, including without limitation encryption using a private key of a public key cryptographic system as described above. Signature may be verified using a verification datum suitable for verification of a secure proof; for instance, where secure proof is enacted by encrypting message using a private key of a public key cryptographic system, verification may include decrypting the encrypted message using the corresponding public key and comparing the decrypted representation to a purported match that was not encrypted; if the signature protocol is well-designed and implemented correctly, this means the ability to create the digital signature is equivalent to possession of the private decryption key and/or device-specific secret. Likewise, if a message making up a mathematical representation of file is well-designed and implemented correctly, any alteration of the file may result in a mismatch with the digital signature; the mathematical representation may be produced using an alteration-sensitive, reliably reproducible algorithm, such as a hashing algorithm as described above. A mathematical representation to which the signature may be compared may be included with signature, for verification purposes; in other embodiments, the algorithm used to produce the mathematical representation may be publicly available, permitting the easy reproduction of the mathematical representation corresponding to any file.

Still viewing FIGS. 1A-B, in some embodiments, digital signatures may be combined with or incorporated in digital certificates. In one embodiment, a digital certificate is a file that conveys information and links the conveyed information to a “certificate authority” that is the issuer of a public key in a public key cryptographic system. Certificate authority in some embodiments contains data conveying the certificate authority's authorization for the recipient to perform a task. The authorization may be the authorization to access a given datum. The authorization may be the authorization to access a given process. In some embodiments, the certificate may identify the certificate authority. The digital certificate may include a digital signature.

With continued reference to FIGS. 1A-B, in some embodiments, a third party such as a certificate authority (CA) is available to verify that the possessor of the private key is a particular entity; thus, if the certificate authority may be trusted, and the private key has not been stolen, the ability of an entity to produce a digital signature confirms the identity of the entity and links the file to the entity in a verifiable way. Digital signature may be incorporated in a digital certificate, which is a document authenticating the entity possessing the private key by authority of the issuing certificate authority and signed with a digital signature created with that private key and a mathematical representation of the remainder of the certificate. In other embodiments, digital signature is verified by comparing the digital signature to one known to have been created by the entity that purportedly signed the digital signature; for instance, if the public key that decrypts the known signature also decrypts the digital signature, the digital signature may be considered verified. Digital signature may also be used to verify that the file has not been altered since the formation of the digital signature.

With continued reference to FIGS. 1A-B, apparatus 100 may include a time-varying storage hash 152 and/or refreshment hash 160, which may have a time limit after which time-varying storage hash 152 and/or refreshment hash 160 is no longer valid. Time limit may be calculated from an initial time, which may be a datum linked to a particular timestamp or other value representing a fixed moment in time, associated with time-varying storage hash 152 and/or refreshment hash 160; initial time may be a time of creation, a time of verification, or other significant time relating to validity of time-varying token. Initial time may include, without limitation, a timestamp, which may include a secure timestamp, and/or a datum linked to a secure timestamp, such as a cryptographic hash of the secure timestamp or the like. As used herein, a “secure timestamp” is an element of data that immutably and verifiably records a particular time, for instance by incorporating a secure proof, cryptographic hash, or other process whereby a party that attempts to modify the time and/or date of the secure timestamp will be unable to do so without the alteration being detected as fraudulent.

With continued reference to FIGS. 1A-B, apparatus 100 may include performing a trusted time evaluation of storage hash 152 and/or refreshment hash 160 by processor 104. As a non-limiting example, secure proof may be generated using a secure timestamp. Generating the secure timestamp may include digitally signing the secure timestamp using any digital signature protocol as described above. In one embodiment authenticity of received data signals is established by utilizing a chain of attestation via one or more attestation schemes (in nonlimiting example, via direct anonymous attestation (DAA)) to verify that a storage hash 152 and/or refreshment hash 160 is an authentic storage hash 152 and/or refreshment hash 160 that has the property of attested time. Generating a secure timestamp may be used to weed out spoofers or “man in the middle attacks.”

Still referring to FIGS. 1A-B, secure timestamp may record the current time in a hash chain. In an embodiment, a hash chain includes a series of hashes, each produced from a message containing a current time stamp (i.e., current at the moment the hash is created) and the previously created hash, which may be combined with one or more additional data; additional data may include a random number, which may be generated for instance using storage hash 152 and/or refreshment hash 160. Additional data may include one or more additional data, including recipe 112, ingredient 124, refreshment preparation style 128 and the like that are received by processor 104. Additional data may be hashed into a Merkle tree or other hash tree, such that a root of the hash tree may be incorporated in an entry in hash chain. It may be computationally infeasible to reverse hash any one entry, particularly in the amount of time during which its currency is important; it may be astronomically difficult to reverse hash the entire chain, rendering illegitimate or fraudulent timestamps referring to the hash chain all but impossible. A purported entry may be evaluated by hashing its corresponding message. In an embodiment, the trusted timestamping procedure utilized is substantially similar to the RFC 3161 standard. In this scenario, the received data signals are locally processed at the listener device by a one-way function, e.g. a hash function, and this hashed output data is sent to a timestamping authority (TSA). The use of secure timestamps as described herein may enable systems and methods as described herein to instantiate attested time. Attested time is the property that a device incorporating a local reference clock may hash data, e.g. refreshment hash 160 and/or storage hash 152, along with the local timestamp of the device. Attested time may additionally incorporate attested identity, attested device architecture and other pieces of information identifying properties of the attesting device. In one embodiment, secure timestamp is generated by a trusted third party (TTP) that appends a timestamp to the hashed output data, applies the TSA private key to sign the hashed output data concatenated to the timestamp, and returns this signed, a.k.a. trusted timestamped data back to the listener device. Alternatively, or additionally, one or more additional participants, such as other verifying nodes, may evaluate secure timestamp, or other party generating secure timestamp and/or perform threshold cryptography with a plurality of such parties, each of which may have performed an embodiment of method to produce a secure timestamp. In an embodiment, [data store] or other parties authenticating digitally signed assertions, devices, and/or user credentials may perform authentication at least in part by evaluating timeliness of entry and/or generation data as assessed against secure timestamp. In an embodiment, secure proof is generated using an attested computing protocol; this may be performed, as a non-limiting example, using any protocol for attested computing as described above.

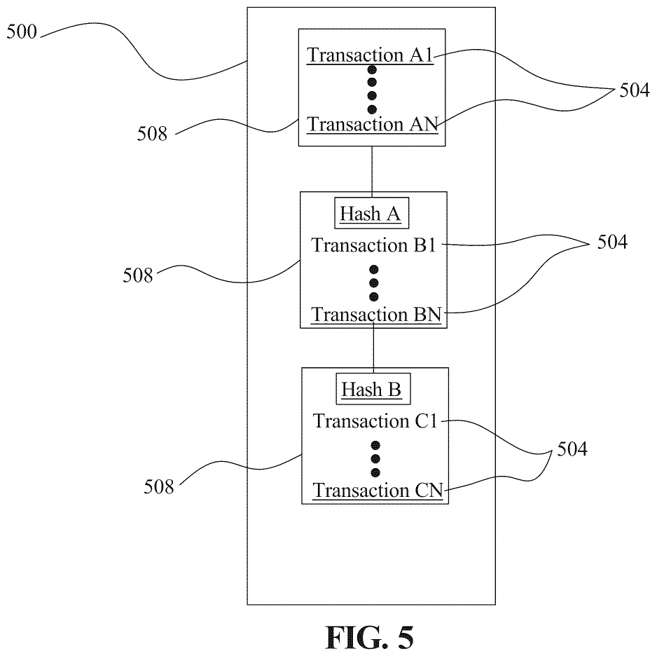

A “storage hash” as used in this disclosure is any ledger 156 utilized to store a hash. A “ledger” as used in this disclosure is a database that is consensually shared, synchronized, and replicated across multiple participants or nodes in different locations, rather than being maintained by a single, centralized authority. Each participant in the network holds an identical copy of the ledger 156, and updates to the ledger 156 are agreed upon through a consensus mechanism, ensuring the data's integrity and security. A ledger 156 may include a temporal sequential listing, including any temporal sequential listing as described below in more detail in reference to FIG. 5. An “immutable sequential listing,” as used in this disclosure, is a data structure that places data entries in a fixed sequential arrangement, such as a temporal sequence of entries and/or blocks thereof, where the sequential arrangement, once established, cannot be altered or reordered. An immutable sequential listing may be, include and/or implement an immutable ledger 156, where data entries that have been posted to the immutable sequential listing cannot be altered. Refreshment option 108 and/or an ingredient 124 may be secured as a refreshment hash 160 within storage hash 152. A “refreshment hash” as used in this disclosure is any mathematical function that transforms a refreshment option 108 (or “message”) into a fixed-size string of characters which may be represented as a sequence of letters and numbers.

With continued reference to FIGS. 1A-B, in an embodiment, methods, systems, and apparatuses described herein may perform or implement one or more aspects of a cryptographic system. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

With continued reference to FIGS. 1A-B, in embodiments of cryptographic systems that are “asymmetric,” either encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

With continued reference to FIGS. 1A-B, in some embodiments, a nutrient badge 184 associated with an updated refreshment option 144 may be secured, recorded, or referenced within storage hash 152 and/or refreshment hash 160, thereby enabling verifiable persistence, traceability, and integrity of badge eligibility determinations. In such embodiments, processor 104 may generate a cryptographic representation of the nutrient badge 184, including associated eligibility criteria 186, scoring parameters, temporal validity information, and identifiers corresponding to the updated refreshment option 144, and may store the cryptographic representation within ledger 156. The nutrient badge 184 may be linked to, derived from, or incorporated into a refreshment hash 160 such that modification of one or more ingredients 124, preparation styles 128, or scoring parameters results in a corresponding change to the hash, thereby rendering badge status tamper-evident. In some embodiments, the nutrient badge 184 stored within storage hash 152 may include temporal attributes defining a duration of validity, refresh interval, or expiration condition, after which badge eligibility must be re-evaluated. This enables processor 104 to associate a time-bound or condition-bound nutrient badge 184 with an updated refreshment option 144, such that badge validity reflects current biological requirements, nutrient intake states, or scoring model outputs rather than historical compliance alone. In such embodiments, ledger-based storage of nutrient badges enables distributed verification of nourishment compliance while preserving the integrity of the underlying recipe-generation and evaluation processes. As used in this disclosure, one or more eligibility criteria 186 refer to a set of definable conditions, thresholds, rules, or constraints evaluated to determine whether an updated refreshment option 144 qualifies for assignment, maintenance, or revocation of a nutrient badge 184. In some embodiments, eligibility criteria 186 are determined as a function of outputs of the dynamic scoring model 180 and may include, without limitation, minimum or maximum nutrient score thresholds, nourishment label ranges, satisfaction of critical daily essential nutrient requirements, nutrient balance constraints, hydration-related thresholds, cumulative intake considerations over a temporal window, or combinations thereof. Eligibility criteria 186 may further incorporate individualized biological requirements derived from an individual biological profile 170, population-level constraints associated with a phenotypic cluster, or contextual conditions such as temporal validity or environmental factors. In some embodiments, eligibility criteria 186 may be static or dynamically adjustable and may be recalibrated over time based on changes to biological utilization data, updated scoring outputs, or revised nutritional objectives. Processor 104 may evaluate the updated refreshment option 144 against the one or more eligibility criteria 186 to assign, withhold, modify, or revoke a nutrient badge 184, wherein satisfaction of the eligibility criteria 186 indicates alignment of the updated refreshment option 144 with defined biological and nutritional requirements as determined by the dynamic scoring model.

With continued reference to FIGS. 1A-B, processor 104 is configured to calculate a nourishment label 164 for updated refreshment option 144. A “nourishment label” as used in this disclosure, is a numerical value assigned to food items to indicate their overall nutritional quality. It is may be calculated based on the presence of key nutrients and ingredient 124, such as by weighing both beneficial and harmful components of the food. A nourishment label 164 be calculated by generating a nutrition score and/or a nourishment score as described in U.S. patent application Ser. No. 16/919,532 filed on Jul. 2, 2020 and entitled “METHODS AND SYSTEMS FOR CALCULATING NUTRITIONAL REQUIREMENTS IN A DISPLAY INTERFACE AND U.S. patent application Ser. No. 18/768,176 filed on Jul. 10, 2024 and entitled “METHODS AND SYSTEMS FOR CALCULATING AN EDIBLE SCORE IN A DISPLAY INTERFACE both of which are herein incorporated by reference in their entirety. Updated refreshment option 144 may be secured within storage hash 152 as a function of a nourishment label 164. In an embodiment, nourishment label 164 may be utilized to sort and retrieve refreshment option 108. For instance and without limitation, a producer may aim to seek a refreshment option 108 that is suitable for a metabolic phenotype and contains a nourishment label 164 with a nourishment label 164 of 78-84. In yet another non-limiting example, a producer may aim to seek a refreshment option 108 that is suitable for cancer prevention and contains a nourishment label 164 somewhere between 55-75. In an embodiment, updated refreshment option 144 may be stored within ledger 156 based on nourishment label 164.

With continued reference to FIGS. 1A-B, in some embodiments, calculation of the nourishment label 164 may further comprise implementing a dynamic scoring model 180 configured to update the nourishment label 164 in response to real-time or near real-time monitoring of nutrient intake 182, hydration status, and biological utilization. In such embodiments, processor 104 may continuously or periodically receive data indicative of nutrients consumed, water intake, or inferred hydration levels associated with an individual biological profile 170, and may adjust weighting factors, scoring thresholds, or nutrient contribution values used to calculate the nourishment label 164 accordingly. The dynamic scoring model 180 may account for cumulative nutrient intake 182 across multiple refreshment options 108 consumed within a defined temporal window, such that the nourishment label 164 reflects not only the intrinsic nutritional properties of a single updated refreshment option 144, but also its contextual contribution toward satisfying critical daily essential nutrient requirements.

In some embodiments, processor 104 may modify the nourishment label 164 as a function of deviations between observed or inferred nutrient and hydration intake and one or more target intake ranges, minimum thresholds, or maximum limits associated with the individual biological profile 170, phenotypic cluster 136, or nourishment indicator 132. For example, where real-time monitoring indicates insufficient water intake, electrolyte imbalance, or disproportionate macronutrient consumption, processor 104 may dynamically adjust scoring parameters to emphasize or de-emphasize corresponding ingredients 124 or nutrient classes in subsequent updated refreshment options 144. In such embodiments, the nourishment label 164 may be recalculated iteratively as new intake data is received, enabling adaptive scoring that reflects changing biological conditions rather than a static nutritional assessment. In further embodiments, the dynamic scoring model may be utilized to generate comparative nourishment labels 164 across a population, subgroup, or phenotypic cluster 136 while simultaneously maintaining individualized scoring outputs tailored to specific biological needs. This enables processor 104 to support real-time nourishment optimization, sorting, eligibility determination, or downstream certification processes based on dynamically evolving nutrient and hydration states.

In some embodiments, a dynamic scoring model is utilized to calculate, update, and recalibrate a nutrient score and/or nourishment label associated with a refreshment option 108 or updated refreshment option 144. As used in this disclosure, a dynamic scoring model is a computational model configured to generate scoring outputs that change over time as a function of newly received biological, nutritional, or contextual data rather than producing a single static score. The dynamic scoring model may include rule-based logic, weighted scoring matrices, constraint-based optimization, one or more machine learning models, or combinations thereof. In embodiments utilizing a machine learning model, the machine learning model may be trained to predict nutrient adequacy, biological alignment, or scoring outcomes based on historical data. Inputs to the dynamic scoring model may include nutrient composition data of ingredients 124, ingredient quantities, preparation styles 128, refreshment source data, individual biological profiles, phenotypic cluster information, critical daily essential nutrient requirements, hydration status, cumulative nutrient intake 182 over a temporal window, and biological utilization or bio extraction indicators. Outputs of the dynamic scoring model may include a nutrient score, a nourishment label 164, adjusted weighting factors, eligibility determinations, or adjustment signals used to modify subsequent updated refreshment options 144. Training data for machine learning embodiments may include historical refreshment options, prior nutrient scores and nourishment labels, aggregated phenotypic data, anonymized biological utilization outcomes, simulated nutrient adequacy scenarios, and prior eligibility determinations, wherein training data may be obtained from curated nutritional databases, public nutrient reference datasets, expert-defined nutrient models, historical system outputs, anonymized user interaction data, synthetic data, or combinations thereof. In such embodiments, the dynamic scoring model is configured to adapt scoring behavior over time while maintaining compliance with defined biological constraints and nutritional requirements. As used in this disclosure, nutrient intake 182 aggregated over a temporal interval refers to a cumulative representation of nutrients consumed by an individual across one or more refreshment options within a defined period of time. The temporal interval may include, without limitation, a meal period, daily interval, rolling time window, user-defined duration, or other discrete or continuous timeframe. In some embodiments, processor 104 aggregates nutrient intake 182 by summing, weighting, or otherwise combining nutrient quantities derived from multiple refreshment options consumed during the temporal interval, wherein aggregation may account for nutrient type, quantity, bioavailability, preparation style, or hydration-related factors. In such embodiments, aggregated nutrient intake 182 may be utilized by the dynamic scoring model to evaluate cumulative biological adequacy relative to critical daily essential nutrient requirements, rather than assessing each refreshment option in isolation. Aggregation over a temporal interval enables the dynamic scoring model to adjust scoring outputs, nourishment labels, eligibility criteria 186, or subsequent refreshment option generation based on whether cumulative intake satisfies, exceeds, or falls short of defined nutritional or biological thresholds. The temporal interval may be static or dynamically adjusted and may be recalculated as new intake data is received.

With continued reference to FIGS. 1A-B, storage hash 152 may include a temporal preparation element 168. A “temporal preparation element” as used in this disclosure is any information relating to preparation time and/or complexity of refreshment option 108. Temporal preparation element 168 may be calculated on a numerical score and/or contain an indication as to complexity of preparation of refreshment option 108. For instance and without limitation, a refreshment option 108 such as a chicken pot pie may contain a temporal preparation element 168 of 7 indicating a time consuming (greater than 1 hour) of preparation time and complexity, whereas a refreshment option 108 such as a mixed green salad may contain a temporal preparation element 168 of 2 indicating a fast (less than 10 minutes) preparation and easy complexity. In an embodiment, temporal preparation element 168 may contain a first score for length of preparation time and a second score for complexity.

With continued reference to FIGS. 1A-B, the embodiments described herein provide one or more technical improvements to computer-based nourishment evaluation and optimization systems. In particular, the disclosed apparatus and methods improve upon conventional static nutritional scoring systems by introducing a dynamically adaptive computational architecture that continuously updates nourishment outputs based on individualized biological profiles, aggregated nutrient intake over temporal intervals, and biological utilization data. Unlike prior systems that generate fixed nutritional scores or recommendations in isolation, the disclosed system integrates real-time and historical data inputs into a dynamic scoring model that recalibrates scoring parameters, nutrient contribution values, and eligibility determinations as system conditions evolve. The disclosed embodiments further improve computer functionality by generating and maintaining structured data objects including nutrient scores, nourishment labels, nutrient badges, and refreshment hashes that are computationally linked and updated in response to changes in ingredient composition, scoring outputs, or biological feedback. By cryptographically generating a refreshment hash as a function of updated refreshment options, nourishment labels, and nutrient badge status, the system provides a tamper-evident mechanism for tracking state changes, versioning nourishment outputs, and enforcing eligibility constraints. This architecture enables efficient storage, retrieval, verification, and comparison of nourishment states across time and system instances, thereby reducing computational redundancy and improving data integrity. Collectively, these features constitute a technical improvement to the functioning of computer systems tasked with evaluating complex, multi-variable nutritional data by enabling adaptive, feedback-driven scoring, constraint-based optimization, and verifiable state management that cannot be achieved through manual processes or conventional static nutritional analysis techniques.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example inputs may include but are not limited to refreshment option 108, ingredient 124, refreshment preparation style 128, phenotypic clusters, recipe 112, visual graphic 120s, prevalence factor 140 and outputs may include updated refreshment option 144, storage hash 152, temporal preparation element 168, refreshment hash 160 and the like.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to sub-categories including but not limited to users with shared characteristics of refreshment option 108, ingredient 124, refreshment preparation style 128, phenotypic clusters and/or shared characteristics of updated refreshment option 144.

Still referring to FIG. 2, a computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. A computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 2, a computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 2, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor 104, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. A computing device, processor 104, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 2, computer, processor 104, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 2, computer, processor 104, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor 104, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor 104, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor 104, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor 104 may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor 104, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor 104, and/or module may use averaging, such as luma or chroma averaging in images, to fill in data units in between original data units.

In some embodiments, and with continued reference to FIG. 2, computing device, processor 104, and/or module may down-sample elements of a training example to a desired lower number of data elements. As a non-limiting example, a high pixel count image may have 256 pixels, however a desired number of pixels may be 128. Processor 104 may down-sample the high pixel count image to convert the 256 pixels into 128 pixels. In some embodiments, processor 104 may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 2, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 2, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 2, computing device, processor 104, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may inputs may include but are not limited to refreshment option 108, ingredient 124, refreshment preparation style 128, phenotypic clusters, recipe 112, visual graphic 120s, prevalence factor 140, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor 104, and/or module may be configured to perform method, method step, sequence of method steps and/or algorithm described in reference to this figure, in any order and with any degree of repetition. For instance, a computing device, processor 104, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor 104, and/or module may perform any step, sequence of steps, or algorithm in parallel, such as simultaneously and/or substantially simultaneously performing a step two or more times using two or more parallel threads, processor 104 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor 104 performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor 104, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 2, machine learning process may include a generative machine learning process. As used in this disclosure, a “generative machine learning process” is a process that automatedly, using a prompt (i.e., input), generates an output consistent with training data; this is in contrast to a non-machine learning software program where outputs are determined in advance by a user and written in a programming language. In some embodiments, generative machine-learning processes may determine patterns and structures from training data and use these patterns and structures to synthesize new data with similar characteristics, as a function of an input. As a non-limiting example, generative machine-learning process may determine patterns and structures from training data of language processing models, augmentation machine-learning model, or any machine-learning models described in the entirety of this disclosure and may use these patterns to synthesize new data, augmented action data as a function of an input, such as but not limited to action data, external data, target data point, or the like.

With continued reference to FIG. 2 generative machine learning processes may synthesize data of different types or domains, including without limitation text, code, images, function data 112, functional signature 124, and/or optimized functional signature 128. Exemplary generative machine learning systems trained on words or word tokens, operant in text domain, include GPT-3, LaMDA, LLAMA, BLOOM, GPT-4, and the like. Exemplary machine learning processes trained on programming language text (i.e., code) include without limitation OpenAI Codex. Exemplary machine learning processes trained on sets of images (for instance with text captions) include Imagen, DALL-E, Midjourney, Adobe Firefly, Stable Diffusion, and the like; image generative machine learning processes, in some cases, may be trained for text-to-image generation and/or neural style transfer. Exemplary generative machine learning processes trained on molecular data include, without limitation, AlphaFold, which may be used for protein structure prediction and drug discovery. Generative machine learning processes trained on audio training data include MusicLM which may be trained on audio waveforms of music correlated with text annotations; music generative machine learning processes, in some cases, may generate new musical samples based on text descriptions. Exemplary generative machine learning processes trained on video include without limitation RunwayML and Make-A-Video by Meta Platforms. Finally, exemplary generative machine learning processes trained using robotic action data include without limitation UniPi from Google Research.

With continued reference to FIG. 2, in some cases a generative machine learning process may include a generative adversarial network (GAN). As used in this disclosure, a “generative adversarial network” is a machine learning process that includes at least two adverse networks configured to synthesize data according to prescribed rules (e.g., rules of a game). In some cases, a generative adversarial network may include a generative and a discriminative network, where the generative network generates candidate data and the discriminative network evaluates the candidate data. An exemplary GAN may be described according to a following game: Each probability space (Ω, μ_{ref}) defines a GAN game. There are two adverse networks: a generator network and a discriminator network. Generator network strategy set is P(Ω), the set of all probability measures μ_{G} on Ω. Discriminator network strategy set is the set of Markov kernels μ_{D}:Ω→P[0, 1], where P[0, 1] is set of probability measures on [0,1]. GAN game may be a zero-sum game, with objective function:

$$L ( {μ}_{G} , {μ}_{D} ) := {𝔼}_{x ∼ {μ}_{ref} , y ∼ {μ}_{D} ( x )} [ ln y ] + {𝔼}_{x ∼ {μ}_{G} , y ∼ {μ}_{D} ( x )} [ ln ( 1 - y ) ] .$$

Generally, generator network may aim to minimize objective, and discriminator network may aim to maximize the objective. Specifically, generator network seeks to approach μ_{G}≈μ_{ref}, said another way, generator network produces candidate data that matches its own output distribution as closely as possible to a reference distribution (provided with training data). Discriminator network outputs a value close to 1 when candidate data appears to be from reference (training data) distribution, and to output a value close to 0 when candidate data looks like it came from generator network distribution. Generally speaking, generative network generates candidates while discriminative network evaluates them, with contest operating in terms of data distributions. In some embodiments, generator network may learn to map from a latent space to a data distribution of interest, while discriminator network may distinguish candidates produced by the generator network from a true data distribution (e.g., training data). In some cases, generator network's training objective is to increase an error rate of discriminator network (i.e., “fool” the discriminator network by producing novel candidates that the discriminator thinks are not synthesized but, instead, are part of training data). In some cases, a known dataset may serve as initial training data for discriminator network. Training may involve presenting discriminator network with samples from training dataset until it achieves acceptable accuracy. In some cases, generator network may be trained on whether the generator network succeeds in fooling discriminator network. A generator network may be seeded with randomized input that is sampled from a predefined latent space (e.g. a multivariate normal distribution). Thereafter, candidates synthesized by generator network may be evaluated by discriminator network. Independent backpropagation procedures may be applied to both networks so that generator network may produce better samples, while discriminator network may become more skilled at flagging synthetic samples. When used for image generation, generator network may be a deconvolutional neural network, and discriminator may be a convolutional neural network.

Still referring to FIG. 2, may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records, entity documents, business documents, inventory documentation, emails, user communications, function data 112, temporal elements, functional signature 124, optimized functional signature 128 and the like. In some embodiments, training sets of an LLM may include information from one or more public or private databases. As a non-limiting example, training sets may include databases associated with an entity. In some embodiments, training sets may include portions of documents associated with the electronic records correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 2, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 2, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 2, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 2, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 2, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 2, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 2, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. A query vector may include an entity's learned representation for comparison to determine attention score. A key vector may include an entity's learned representation for determining the entity's relevance and attention weight. A value vector may include data used to generate output representations. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 2, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 2, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 2, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 2, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 2, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 2, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 2, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 2, decoder may take this output and add it to the decoder inputs. Decoder may continue decoding until a token is predicted. Decoder may stop decoding once it predicts an end token.

Continuing to refer to FIG. 2, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 2, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like.

With continued reference to FIG. 2, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

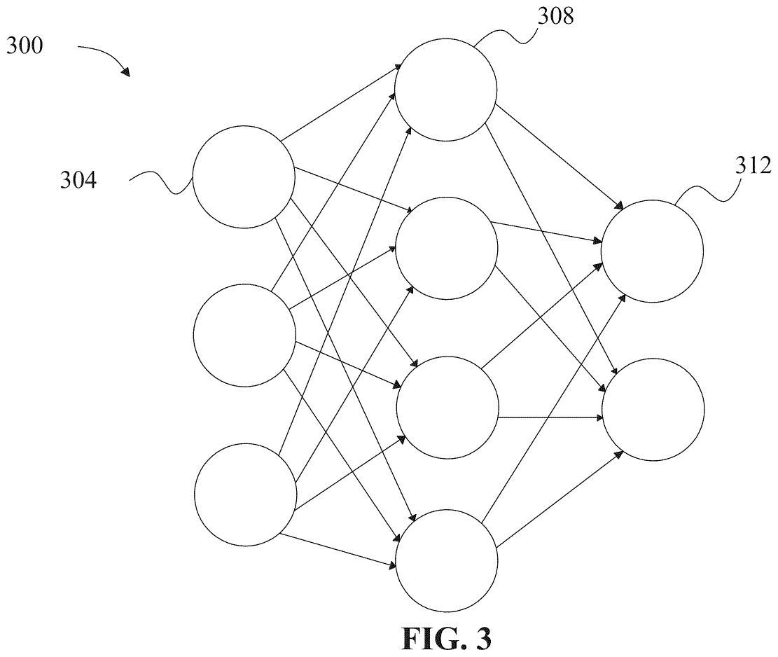

Referring now to FIG. 3, an exemplary embodiment of neural network 3 is illustrated. A neural network 3 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes %% 04, one or more intermediate layers %% 08, and an output layer of nodes %% 12. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

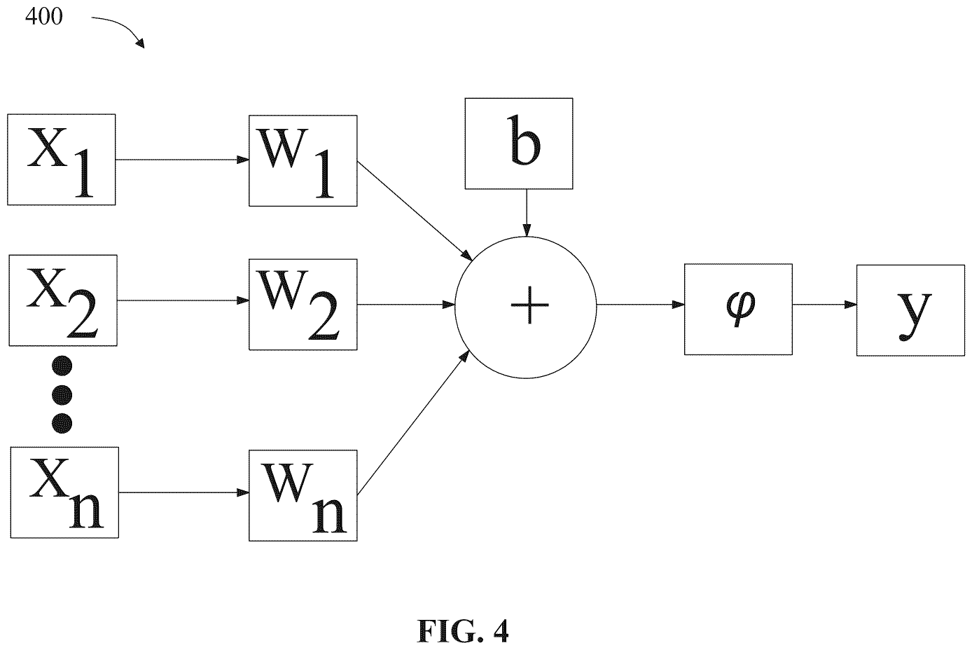

Referring now to FIG. 4, an exemplary embodiment of a node 400 of a neural network is illustrated. A node may include, without limitation a plurality of inputs x, that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form ƒ(x)=1/1−e^{-x} given input x, a tan h (hyperbolic tangent) function, of the form e^{x}−e^{-x}/e^{x}+e^{-x}, a tan h derivative function such as ƒ(x)=tan h²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tan h(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Referring now to FIG. 5, an exemplary embodiment of an immutable sequential listing 500 is illustrated. Data elements are listing in immutable sequential listing 500; data elements may include any form of data, including textual data, image data, encrypted data, cryptographically hashed data, and the like. Data elements may include, without limitation, one or more at least a digitally signed assertions. In one embodiment, a digitally signed assertion 504 is a collection of textual data signed using a secure proof as described in further detail below; secure proof may include, without limitation, a digital signature as described above. Collection of textual data may contain any textual data, including without limitation American Standard Code for Information Interchange (ASCII), Unicode, or similar computer-encoded textual data, any alphanumeric data, punctuation, diacritical mark, or any character or other marking used in any writing system to convey information, in any form, including any plaintext or cyphertext data; in an embodiment, collection of textual data may be encrypted, or may be a hash of other data, such as a root or node of a Merkle tree or hash tree, or a hash of any other information desired to be recorded in some fashion using a digitally signed assertion 504. In an embodiment, collection of textual data states that the owner of a certain transferable item represented in a digitally signed assertion 504 register is transferring that item to the owner of an address. A digitally signed assertion 504 may be signed by a digital signature created using the private key associated with the owner's public key, as described above.

Still referring to FIG. 5, a digitally signed assertion 504 may describe a transfer of virtual currency, such as crypto-currency and/or cryptographic hash as described below. The virtual currency may be a digital currency. Item of value may be a transfer of trust, for instance represented by a statement vouching for the identity or trustworthiness of the first entity. Item of value may be an interest in a fungible negotiable financial instrument representing ownership in a public or private corporation, a creditor relationship with a governmental body or a corporation, rights to ownership represented by an option, derivative financial instrument, commodity, debt-backed security such as a bond or debenture or other security as described in further detail below. A resource may be a physical machine e.g. a ride share vehicle or any other asset. A digitally signed assertion 504 may describe the transfer of a physical good; for instance, a digitally signed assertion 504 may describe the sale of a product. In some embodiments, a transfer nominally of one item may be used to represent a transfer of another item; for instance, a transfer of virtual currency may be interpreted as representing a transfer of an access right; conversely, where the item nominally transferred is something other than virtual currency, the transfer itself may still be treated as a transfer of virtual currency, having value that depends on many potential factors including the value of the item nominally transferred and the monetary value attendant to having the output of the transfer moved into a particular user's control. The item of value may be associated with a digitally signed assertion 504 by means of an exterior protocol, such as the COLORED COINS created according to protocols developed by The Colored Coins Foundation, the MASTERCOIN protocol developed by the Mastercoin Foundation, or the ETHEREUM platform offered by the Stiftung Ethereum Foundation of Baar, Switzerland, the Thunder protocol developed by Thunder Consensus, or any other protocol.

Still referring to FIG. 5, in one embodiment, an address is a textual datum identifying the recipient of virtual currency or another item of value in a digitally signed assertion 504. In some embodiments, address is linked to a public key, the corresponding private key of which is owned by the recipient of a digitally signed assertion 504. For instance, address may be the public key. Address may be a representation, such as a hash, of the public key. Address may be linked to the public key in memory of a computing device, for instance via a “wallet shortener” protocol. Where address is linked to a public key, a transferee in a digitally signed assertion 504 may record a subsequent a digitally signed assertion 504 transferring some or all of the value transferred in the first a digitally signed assertion 504 to a new address in the same manner. A digitally signed assertion 504 may contain textual information that is not a transfer of some item of value in addition to, or as an alternative to, such a transfer. For instance, as described in further detail below, a digitally signed assertion 504 may indicate a confidence level associated with a distributed storage node as described in further detail below.

In an embodiment, and still referring to FIG. 5 immutable sequential listing 500 records a series of at least a posted content in a way that preserves the order in which the at least a posted content took place. Temporally sequential listing may be accessible at any of various security settings; for instance, and without limitation, temporally sequential listing may be readable and modifiable publicly, may be publicly readable but writable only by entities and/or devices having access privileges established by password protection, confidence level, or any device authentication procedure or facilities described herein, or may be readable and/or writable only by entities and/or devices having such access privileges. Access privileges may exist in more than one level, including, without limitation, a first access level or community of permitted entities and/or devices having ability to read, and a second access level or community of permitted entities and/or devices having ability to write; first and second community may be overlapping or non-overlapping. In an embodiment, posted content and/or immutable sequential listing 500 may be stored as one or more zero knowledge sets (ZKS), Private Information Retrieval (PIR) structure, or any other structure that allows checking of membership in a set by querying with specific properties. Such database may incorporate protective measures to ensure that malicious actors may not query the database repeatedly in an effort to narrow the members of a set to reveal uniquely identifying information of a given posted content.

Still referring to FIG. 5, immutable sequential listing 500 may preserve the order in which the at least a posted content took place by listing them in chronological order; alternatively or additionally, immutable sequential listing 500 may organize digitally signed assertions 504 into sub-listings 508 such as “blocks” in a blockchain, which may be themselves collected in a temporally sequential order; digitally signed assertions 504 within a sub-listing 508 may or may not be temporally sequential. The ledger 156 may preserve the order in which at least a posted content took place by listing them in sub-listings 508 and placing the sub-listings 508 in chronological order. The immutable sequential listing 500 may be a distributed, consensus-based ledger 156, such as those operated according to the protocols promulgated by Ripple Labs, Inc., of San Francisco, Calif., or the Stellar Development Foundation, of San Francisco, Calif, or of Thunder Consensus. In some embodiments, the ledger 156 is a secured ledger 156; in one embodiment, a secured ledger 156 is a ledger 156 having safeguards against alteration by unauthorized parties. The ledger 156 may be maintained by a proprietor, such as a system administrator on a server, that controls access to the ledger 156; for instance, the user account controls may allow contributors to the ledger 156 to add at least a posted content to the ledger 156, but may not allow any users to alter at least a posted content that have been added to the ledger 156. In some embodiments, ledger 156 is cryptographically secured; in one embodiment, a ledger 156 is cryptographically secured where each link in the chain contains encrypted or hashed information that makes it practically infeasible to alter the ledger 156 without betraying that alteration has taken place, for instance by requiring that an administrator or other party sign new additions to the chain with a digital signature. Immutable sequential listing 500 may be incorporated in, stored in, or incorporate, any suitable data structure, including without limitation any database, datastore, file structure, distributed hash table, directed acyclic graph or the like. In some embodiments, the timestamp of an entry is cryptographically secured and validated via trusted time, either directly on the chain or indirectly by utilizing a separate chain. In one embodiment the validity of timestamp is provided using a time stamping authority as described in the RFC 3161 standard for trusted timestamps, or in the ANSI ASC x9.95 standard. In another embodiment, the trusted time ordering is provided by a group of entities collectively acting as the time stamping authority with a requirement that a threshold number of the group of authorities sign the timestamp.

In some embodiments, and with continued reference to FIG. 5, immutable sequential listing 500, once formed, may be inalterable by any party, no matter what access rights that party possesses. For instance, immutable sequential listing 500 may include a hash chain, in which data is added during a successive hashing process to ensure non-repudiation. Immutable sequential listing 500 may include a block chain. In one embodiment, a block chain is immutable sequential listing 500 that records one or more new at least a posted content in a data item known as a sub-listing 508 or “block.” An example of a block chain is the BITCOIN block chain used to record BITCOIN transactions and values. Sub-listings 508 may be created in a way that places the sub-listings 508 in chronological order and link each sub-listing 508 to a previous sub-listing 508 in the chronological order so that any computing device may traverse the sub-listings 508 in reverse chronological order to verify any at least a posted content listed in the block chain. Each new sub-listing 508 may be required to contain a cryptographic hash describing the previous sub-listing 508. In some embodiments, the block chain contains a single first sub-listing 508 sometimes known as a “genesis block.”

Still referring to FIG. 5, the creation of a new sub-listing 508 may be computationally expensive; for instance, the creation of a new sub-listing 508 may be designed by a “proof of work” protocol accepted by all participants in forming the immutable sequential listing 500 to take a powerful set of computing devices a certain period of time to produce. Where one sub-listing 508 takes less time for a given set of computing devices to produce the sub-listing 508 protocol may adjust the algorithm to produce the next sub-listing 508 so that it will require more steps; where one sub-listing 508 takes more time for a given set of computing devices to produce the sub-listing 508 protocol may adjust the algorithm to produce the next sub-listing 508 so that it will require fewer steps. As an example, protocol may require a new sub-listing 508 to contain a cryptographic hash describing its contents; the cryptographic hash may be required to satisfy a mathematical condition, achieved by having the sub-listing 508 contain a number, called a nonce, whose value is determined after the fact by the discovery of the hash that satisfies the mathematical condition. Continuing the example, the protocol may be able to adjust the mathematical condition so that the discovery of the hash describing a sub-listing 508 and satisfying the mathematical condition requires more or less steps, depending on the outcome of the previous hashing attempt. Mathematical condition, as an example, might be that the hash contains a certain number of leading zeros and a hashing algorithm that requires more steps to find a hash containing a greater number of leading zeros, and fewer steps to find a hash containing a lesser number of leading zeros. In some embodiments, production of a new sub-listing 508 according to the protocol is known as “mining.” The creation of a new sub-listing 508 may be designed by a “proof of stake” protocol as will be apparent to those skilled in the art upon reviewing the entirety of this disclosure.

Continuing to refer to FIG. 5, in some embodiments, protocol also creates an incentive to mine new sub-listings 508. The incentive may be financial; for instance, successfully mining a new sub-listing 508 may result in the person or entity that mines the sub-listing 508 receiving a predetermined amount of currency. The currency may be fiat currency. Currency may be cryptocurrency as defined below. In other embodiments, incentive may be redeemed for particular products or services; the incentive may be a gift certificate with a particular business, for instance. In some embodiments, incentive is sufficiently attractive to cause participants to compete for the incentive by trying to race each other to the creation of sub-listings 508. Each sub-listing 508 created in immutable sequential listing 500 may contain a record or at least a posted content describing one or more addresses that receive an incentive, such as virtual currency, as the result of successfully mining the sub-listing 508.

With continued reference to FIG. 5, where two entities simultaneously create new sub-listings 508, immutable sequential listing 500 may develop a fork; protocol may determine which of the two alternate branches in the fork is the valid new portion of the immutable sequential listing 500 by evaluating, after a certain amount of time has passed, which branch is longer. “Length” may be measured according to the number of sub-listings 508 in the branch. Length may be measured according to the total computational cost of producing the branch. Protocol may treat only at least a posted content contained the valid branch as valid at least a posted content. When a branch is found invalid according to this protocol, at least a posted content registered in that branch may be recreated in a new sub-listing 508 in the valid branch; the protocol may reject “double spending” at least a posted content that transfer the same virtual currency that another at least a posted content in the valid branch has already transferred. As a result, in some embodiments the creation of fraudulent at least a posted content requires the creation of a longer immutable sequential listing 500 branch by the entity attempting the fraudulent at least a posted content than the branch being produced by the rest of the participants; as long as the entity creating the fraudulent at least a posted content is likely the only one with the incentive to create the branch containing the fraudulent at least a posted content, the computational cost of the creation of that branch may be practically infeasible, guaranteeing the validity of all at least a posted content in the immutable sequential listing 500.

Still referring to FIG. 5, additional data linked to at least a posted content may be incorporated in sub-listings 508 in the immutable sequential listing 500; for instance, data may be incorporated in one or more fields recognized by block chain protocols that permit a person or computer forming a at least a posted content to insert additional data in the immutable sequential listing 500. In some embodiments, additional data is incorporated in an unspendable at least a posted content field. For instance, the data may be incorporated in an OP_RETURN within the BITCOIN block chain. In other embodiments, additional data is incorporated in one signature of a multi-signature at least a posted content. In an embodiment, a multi-signature at least a posted content is at least a posted content to two or more addresses. In some embodiments, the two or more addresses are hashed together to form a single address, which is signed in the digital signature of the at least a posted content. In other embodiments, the two or more addresses are concatenated. In some embodiments, two or more addresses may be combined by a more complicated process, such as the creation of a Merkle tree or the like. In some embodiments, one or more addresses incorporated in the multi-signature at least a posted content are typical crypto-currency addresses, such as addresses linked to public keys as described above, while one or more additional addresses in the multi-signature at least a posted content contain additional data related to the at least a posted content; for instance, the additional data may indicate the purpose of the at least a posted content, aside from an exchange of virtual currency, such as the item for which the virtual currency was exchanged. In some embodiments, additional information may include network statistics for a given node of network, such as a distributed storage node, e.g. the latencies to nearest neighbors in a network graph, the identities or identifying information of neighboring nodes in the network graph, the trust level and/or mechanisms of trust (e.g. certificates of physical encryption keys, certificates of software encryption keys, (in non-limiting example certificates of software encryption may indicate the firmware version, manufacturer, hardware version and the like), certificates from a trusted third party, certificates from a decentralized anonymous authentication procedure, and other information quantifying the trusted status of the distributed storage node) of neighboring nodes in the network graph, IP addresses, GPS coordinates, and other information informing location of the node and/or neighboring nodes, geographically and/or within the network graph. In some embodiments, additional information may include history and/or statistics of neighboring nodes with which the node has interacted. In some embodiments, this additional information may be encoded directly, via a hash, hash tree or other encoding.

With continued reference to FIG. 5, in some embodiments, virtual currency is traded as a crypto-currency. In one embodiment, a crypto-currency is a digital, currency such as Bitcoins, Peercoins, Namecoins, and Litecoins. Crypto-currency may be a clone of another crypto-currency. The crypto-currency may be an “alt-coin.” Crypto-currency may be decentralized, with no particular entity controlling it; the integrity of the crypto-currency may be maintained by adherence by its participants to established protocols for exchange and for production of new currency, which may be enforced by software implementing the crypto-currency. Crypto-currency may be centralized, with its protocols enforced or hosted by a particular entity. For instance, crypto-currency may be maintained in a centralized ledger 156, as in the case of the XRP currency of Ripple Labs, Inc., of San Francisco, Calif. In lieu of a centrally controlling authority, such as a national bank, to manage currency values, the number of units of a particular crypto-currency may be limited; the rate at which units of crypto-currency enter the market may be managed by a mutually agreed-upon process, such as creating new units of currency when mathematical puzzles are solved, the degree of difficulty of the puzzles being adjustable to control the rate at which new units enter the market. Mathematical puzzles may be the same as the algorithms used to make productions of sub-listings 508 in a block chain computationally challenging; the incentive for producing sub-listings 508 may include the grant of new crypto-currency to the miners. Quantities of crypto-currency may be exchanged using at least a posted content as described above.

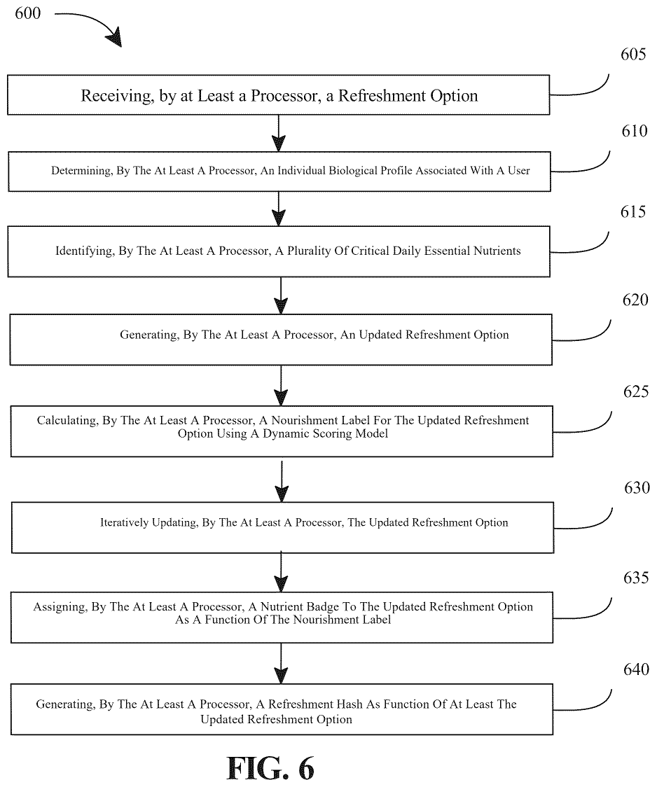

Referring now to FIG. 6, an exemplary embodiment of a method 600 of generating a refreshment hash 160 is illustrated. At step 605, processor 104 receives a refreshment option 108. Refreshment option 108 includes any refreshment option 108 as described above in more detail in reference to FIGS. 1-5.

With continued reference to FIG. 6, at step 610, processor 104 determines an individual biological profile associated with a user. The individual biological profile is determined as a function of a phenotypic cluster. The individual biological profile and phenotypic cluster include any individual biological profile and phenotypic cluster as described above in more detail in reference to FIGS. 1-5.

With continued reference to FIG. 6, at step 615, processor 104 identifies a plurality of critical daily essential nutrients as a function of the determined individual biological profile. The plurality of critical daily essential nutrients includes any critical daily essential nutrients as described above in more detail in reference to FIGS. 1-5.

With continued reference to FIG. 6, at step 620, processor 104 generates an updated refreshment option 144 by adjusting at least an ingredient 124 of the refreshment option 108 as a function of the individual biological profile and the plurality of critical daily essential nutrients. Adjusting at least an ingredient 124 includes any adjustment as described above in more detail in reference to FIGS. 1-5.

With continued reference to FIG. 6, at step 625, processor 104 calculates a nourishment label 164 for the updated refreshment option 144 using a dynamic scoring model. The dynamic scoring model is configured to evaluate a nutrient intake. Nourishment label 164 and the dynamic scoring model include any nourishment label and dynamic scoring model as described above in more detail in reference to FIGS. 1-5.

With continued reference to FIG. 6, at step 630, processor 104 iteratively updates the updated refreshment option 144 by evaluating biological utilization data associated with consumption of the updated refreshment option 144. Biological utilization data includes any biological utilization data as described above in more detail in reference to FIGS. 1-5.

With continued reference to FIG. 6, at step 635, processor 104 assigns a nutrient badge to the updated refreshment option 144 as a function of the nourishment label 164 and one or more eligibility criteria determined as a function of the dynamic scoring model. The nutrient badge and eligibility criteria include any nutrient badge and eligibility criteria as described above in more detail in reference to FIGS. 1-5.

With continued reference to FIG. 6, at step 640, processor 104 generates a refreshment hash 160 as a function of at least the updated refreshment option 144, the nourishment label 164, and the nutrient badge. The refreshment hash 160 may be generated utilizing any hashing or cryptographic methodology as described above in more detail in reference to FIGS. 1-5. It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

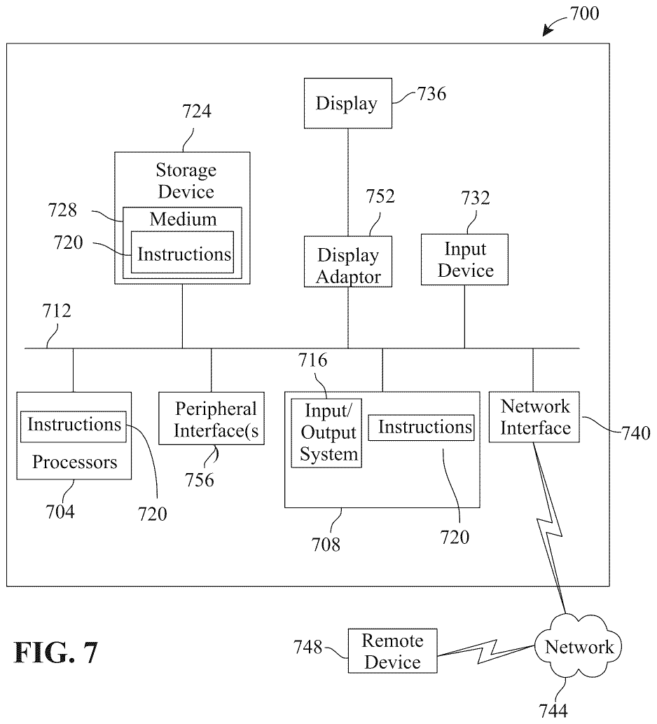

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor 104, such as without limitation a processor 104 incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor 104, digital signal processor 104 (DSP), Field Programmable Gate Array (FPGA), Complex Programmable Logic Device (CPLD), Graphical Processing Unit (GPU), general purpose GPU, Tensor Processing Unit (TPU), analog or mixed signal processor 104, Trusted Platform Module (TPM), a floating point unit (FPU), system on module (SOM), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for generating a refreshment hash, wherein the apparatus comprises:
at least a processor; and
a memory communicatively connected to the at least a processor, wherein the memory containing instructions configuring the at least a processor to:
receive a refreshment option;
determine an individual biological profile associated with a user, wherein the individual biological profile is determined as a function of a phenotypic cluster;
identify a plurality of critical daily essential nutrients as a function of the determined individual biological profile;
generate an updated refreshment option by adjusting at least an ingredient of the refreshment option as a function of the individual biological profile and the plurality of critical daily essential nutrients;
calculate a nourishment label for the updated refreshment option using a dynamic scoring model, wherein the dynamic scoring model is configured to evaluate a nutrient intake;
iteratively update the updated refreshment option by evaluating biological utilization data associated with consumption of the updated refreshment option;
assign a nutrient badge to the updated refreshment option as a function of the nourishment label and one or more eligibility criteria determined as a function of the dynamic scoring model; and
generate a refreshment hash as function of at least the updated refreshment option, the nourishment label, and the nutrient badge.

2. The apparatus of claim 1, wherein the individual biological profile comprises at least one of biochemical markers, metabolic characteristics, hydration status, nutrient absorption efficiency, activity level, and inferred physiological state.

3. The apparatus of claim 1, wherein the nutrient badge is generated for at least one of an individual user, a phenotypic cluster, and a population subgroup.

4. The apparatus of claim 1, further configured to secure the nutrient badge within a storage hash recorded in a ledger.

5. The apparatus of claim 4, wherein adjusting the at least an ingredient results in a corresponding modification to the storage hash.

6. The apparatus of claim 1, wherein the nutrient badge is revoked when the updated refreshment option no longer satisfies at least one eligibility criterion.

7. The apparatus of claim 1, wherein the individual biological profile includes a predicted biological state generated by a machine learning model.

8. The apparatus of claim 1, wherein the nutrient intake is aggregated over a temporal interval.

9. The apparatus of claim 1, wherein the at least a processor is further configured to generate a nutrient score as a function of the updated refreshment option.

10. The apparatus of claim 9, wherein generating the nutrient score further comprises:
identifying a plurality of nutrients as a function of the updated refreshment option;
determining a contribution value for each identified nutrient;
applying one or more weighting factors to the contribution values as a function of the individual biological profile and critical essential nutrient requirements; and
aggregating the weighted contribution values to generate a composite nutrient score.

11. A method of generating a refreshment hash, wherein the method comprises:
receiving, by at least a processor, a refreshment option;
determining, by the at least a processor, an individual biological profile associated with a user, wherein the individual biological profile is determined as a function of a phenotypic cluster;
identifying, by the at least a processor, a plurality of critical daily essential nutrients as a function of the determined individual biological profile;
generating, by the at least a processor, an updated refreshment option by adjusting at least an ingredient of the refreshment option as a function of the individual biological profile and the plurality of critical daily essential nutrients;
calculating, by the at least a processor, a nourishment label for the updated refreshment option using a dynamic scoring model, wherein the dynamic scoring model is configured to evaluate a nutrient intake;
iteratively updating, by the at least a processor, the updated refreshment option by evaluating biological utilization data associated with consumption of the updated refreshment option;
assigning, by the at least a processor, a nutrient badge to the updated refreshment option as a function of the nourishment label and one or more eligibility criteria determined as a function of the dynamic scoring model; and
generating, by the at least a processor, a refreshment hash as function of at least the updated refreshment option, the nourishment label, and the nutrient badge.

12. The method of claim 11, wherein the individual biological profile comprises at least one of biochemical markers, metabolic characteristics, hydration status, nutrient absorption efficiency, activity level, and inferred physiological state.

13. The method of claim 11, wherein the nutrient badge is generated for at least one of an individual user, a phenotypic cluster, and a population subgroup.

14. The method of claim 11, further configured to secure the nutrient badge within a storage hash recorded in a ledger.

15. The method of claim 14, wherein adjusting the at least an ingredient results in a corresponding modification to the storage hash.

16. The method of claim 11, wherein the nutrient badge is revoked when the updated refreshment option no longer satisfies at least one eligibility criterion.

17. The method of claim 11, wherein the individual biological profile includes a predicted biological state generated by a machine learning model.

18. The method of claim 11, wherein the nutrient intake is aggregated over a temporal interval.

19. The method of claim 11, wherein the at least a processor is further configured to generate a nutrient score as a function of the updated refreshment option.

20. The method of claim 19, wherein generating the nutrient score further comprises:
identifying a plurality of nutrients as a function of the updated refreshment option;
determining a contribution value for each identified nutrient;
applying one or more weighting factors to the contribution values as a function of the individual biological profile and critical essential nutrient requirements; and
aggregating the weighted contribution values to generate a composite nutrient score.
